(19) United States
(12) Patent Application Publication
(10) Pub. No.: US 2026/0364047 A1
(43) Pub. Date: Nov. 19, 2026

(54) METHODS OF SWITCHING NEUROPSYCHIATRIC MEDICATIONS USING ULOTARONT

(71) Applicant: Sumitomo Pharma America, Inc., Marlborough, MA (US)

(72) Inventors: David CRANDALL, Marlborough, MA (US); Heather DWORAK, Marlborough, MA (US); Michael TOCCO, Marlborough, MA (US); Courtney ZENI, Marlborough, MA (US)

(73) Assignee: Sumitomo Pharma America, Inc., Marlborough, MA (US)

(21) Appl. No.: 19/129,705

(22) PCT Filed: Nov. 14, 2023
(86) PCT No.: PCT/US2023/079576

§ 371 Date: May 14, 2025

Related U.S. Application Data
(60) Provisional application No. 63/383,774, filed on Nov. 15, 2022.

(51) Int. Cl. A61K 31/381; A61K 45/06; A61P 25/18

(52) U.S. Cl. CPC A61K 31/381; A61K 45/06; A61P 25/18

(57) ABSTRACT

Neuropsychiatric treatments, including methods and regimens for switching neuropsychiatric medications based on ulotaront administration.

### CROSS-REFERENCE TO RELATED APPLICATIONS

The present application claims priority to U.S. provisional application 63/383,774, filed Nov. 15, 2022, the contents of which are incorporated herein by reference in their entirety.

### FIELD

The present disclosure relates to neuropsychiatric medications, and to methods and regimens for switching medications based on ulotaront administration.

### BACKGROUND

Neuropsychiatric medications, particularly antipsychotics, are the first-line agents for managing schizophrenia and related disorders. As with other chronic illnesses, clinicians must often decide whether to switch a patient's current medication to try to improve treatment response or to reduce intolerable side effects and improve quality of life and functioning. Antipsychotic agents are commonly classified as typical antipsychotics or atypical antipsychotics. The atypical antipsychotics differ from typical antipsychotics by their increased binding to 5-HT2a receptors and by the degree and reversibility in D2 receptor binding.

Switching between antipsychotic medications is common in the treatment of schizophrenia as the physician seeks a satisfactory treatment, but such switching has been shown to be associated with poorer clinical and economic outcomes (Faries 2009). There may be many reasons for switching antipsychotic treatment, including unsatisfactory response to current treatment, unwanted side effects, comorbid physical and psychiatric conditions, and patient request. Clinically warranted switches can provide benefits by enhancing treatment effectiveness, tolerability, and overall acceptance by patients (Weiden 2003). Studies have shown that up to one-third of outpatients with schizophrenia in the United States switch antipsychotic therapy within one year (Weiden 2006, Buckley 2007). According to most empirically based criteria (Kinon 2000), successful switching paradigms involve gradual discontinuation of the original antipsychotic drug upon initiation of the new treatment. Individual patient characteristics, as well as the binding profile and dose level of the original antipsychotic, can influence the appropriate duration for successful discontinuation (Buckley 2007; Cerovecki 2013; Takeuchi 2018).

During a switch, patients often experience adverse events. These events often result from the complex pharmacology of neuropsychiatric drugs and their targeting of various receptor subtypes (e.g., D2, 5-HT2A, M1, α1, H1) with varying degrees of affinity. For example, switching from low to high affinity D2 antagonists (e.g., quetiapine to haloperidol) can result in dyskinesias, parkinsonism, akathisia and acute dystonias. Conversely, when switching from a high to a low affinity D2 antagonist, patients may experience breakthrough psychosis, withdrawal dyskinesias and akathisia. Other examples can be cited. See, e.g., Ostuzzi 2022, Ayyagari 2020, Fulone 2021.

Several techniques are used for switching between different conventional antipsychotic drugs. One method of switching involves cross titration where the atypical or typical antipsychotic dose is gradually reduced while the dose of the replacement antipsychotic is gradually increased to the therapeutic dose. A second method is to continue with the dosing of the typical or typical antipsychotic dosing while slowly titrating the replacement antipsychotic dose to near the therapeutic dose, then gradually reducing and ultimately stopping the dosing of the first antipsychotic agent, and then titrating the final dose of the replacement antipsychotic to the therapeutic dose. Both of these techniques have been used to switch between different conventional antipsychotics. With cross titration, switching between different conventional antipsychotics selection of the cross titration is difficult because of different pharmacokinetics between the antipsychotic agents. With the second method, there will be significant overlap of the two antipsychotics with a likelihood of adverse effects during the switch. There is also the risk that the planned discontinuation of the first antipsychotic never takes place or a therapeutic dose of second antipsychotic is not reached. A technique is to directly switch from one conventional antipsychotic to another, but this can result in drug interactions and withdrawal symptoms. This technique can be used where both antipsychotics have a long half-life and no anticholinergic effects. In the case of the first two techniques, the switch may take several months which can lead to patient compliance issues which can result in inadequate treatment of the psychosis or adverse events. (Stahl 2020).

A new neuropsychiatric medication that has been demonstrated to improve symptoms of psychosis in schizophrenia, ulotaront, has a mode of action which is different from the current class of conventional antipsychotics in that it does not involve binding at the D2 receptor but has a mode of action which relies primarily on its trace amine-associated receptor 1 (TAAR1) agonist activity. Unlike the current class of conventional antipsychotics, ulotaront does not significantly interact with receptors known to confer side effects of atypical antipsychotics (e.g., D2, α1, M1, H1, 5-HT2c). Each of the conventional antipsychotics, in addition to binding to the D2 receptor, binds to other receptors with each having its own unique binding profile.

A need exists for a method of switching patients from the conventional antipsychotics to a new class of medication including ulotaront and its pharmaceutically acceptable salts, to provide a more effective and/or tolerable long-term treatment, where adverse events from switching are minimized or potentially eliminated.

There is also a strong need for more clinical research on this topic and for additional resources and medications to help clinicians implement optimal switching strategies for both safety and efficacy.

### SUMMARY

In one embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose, and wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the first ulotaront tapering dose is optionally less than the final ulotaront dose, and (ii) the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first NM tapering dose is the same as the treatment NM dose, and the first ulotaront tapering dose is optionally less than the final ulotaront dose.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period, (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the first NM tapering dose is the same as the treatment NM dose, and (ii) the first ulotaront tapering dose is optionally less than the final ulotaront dose.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication fails to adequately improve the neuropsychiatric disorder; and (ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication fails adequately to improve the neuropsychiatric disorder; and (ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication affects a target receptor in the subject; and (ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication affects a target receptor in the subject; and (ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication produces one or more adverse events in the subject; and (ii) the ulotaront or pharmaceutically acceptable salt thereof does not produce one or more of the adverse events in the subject, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period, (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication produces one or more adverse events in the subject; and (ii) the ulotaront or pharmaceutically acceptable salt thereof does not produce one or more of the adverse events in the subject, optionally wherein the subject takes the neuropsychiatric medication for at least two weeks during the tapering period.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

FIG. 1 is a schematic timeline illustrating the design of the study reported in Example 2.

FIG. 2 is a schematic timeline illustrating the design of the study reported in Example 3.

### DETAILED DESCRIPTION

All published documents cited herein are hereby incorporated herein by reference in their entirety.

### USE OF TERMS

As used herein, the singular forms “a”, “an” and “the” are intended to include the plural forms as well, unless the context clearly indicates otherwise.

Unless otherwise specified, the word “includes” (or any variation thereon, e.g., “include”, “including”, etc.) is intended to be open-ended. For example, “A includes 1, 2 and 3” means that A includes, but is not limited to, 1, 2 and 3.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or particular numerical values are specified, it will be understood that a range can be defined by selectively combining any of the lower end variables, upper end variables, and particular numerical values that is mathematically possible. When ranges are stated as extending from one endpoint to another endpoint, it will be understood that the two endpoints are included in the range. However, it will also be understood that a from/to range also includes an embodiment in which the range is defined as between the two specified endpoints, and that the term “between” can be substituted for the “from/to” language to omit the endpoints from the range.

The present disclosure describes various embodiments. A person of ordinary skill in the art reviewing the disclosure will readily recognize that various embodiments can be combined in any variation. For example, embodiments of the disclosure include treatment of various disorders, patient populations, administrations of dosage forms, at various dosages, minimization of various adverse events, and improvements in various efficacy measures, etc. Any combinations of various embodiments are within the scope of the disclosure.

When published test methodologies and diagnostic instruments are referred to herein, it will be understood that the test methodology or diagnostic instrument is performed based on the version in effect on Oct. 1, 2022, unless otherwise stated to the contrary herein. This is true even when the methodology or instrument is defined herein based on a publication reporting an earlier version.

Unless defined otherwise, all technical and scientific terms used herein have the same meaning as commonly understood by one of ordinary skill in the art to which this disclosure belongs.

### DEFINITIONS

As used herein, “administering” or “administration” of ulotaront, or a pharmaceutically acceptable salt thereof, encompasses the delivery to a subject of ulotaront, or a pharmaceutically acceptable salt thereof, or a prodrug or other pharmaceutically acceptable derivative thereof, using any suitable formulation or route of administration, e.g., as described herein.

An “AE” or “adverse event” is any untoward medical occurrence associated with the use of a drug in humans, whether or not considered drug related. Those untoward medical occurrences that occur after first administration of study drug are considered AEs. An AE can, therefore, be any unfavorable and unintended sign (including an abnormal laboratory finding), symptom, or disease occurring after the administration of a medicinal (investigational) product, whether or not considered related to the medicinal (investigational) product. AEs may include the onset of new illness and the exacerbation of pre-existing conditions. A “mild” AE, as used herein and throughout this document, refers to “ordinarily transient symptoms that do not influence performance of a subject's daily activities. Other treatment is not ordinarily indicated.” A “moderate” AE, as used herein and throughout this document, refers to “marked symptoms sufficient to make the subject uncomfortable. Moderate influence on performance of subject's daily activities. Other treatment may be necessary.” A “severe” AE, as used herein and throughout this document, refers to “symptoms that cause considerable discomfort. Substantial influence on subject's daily activities. May be unable to continue the study, and other treatment may be necessary.”

As disclosed herein, the methods of the current disclosure can be undertaken without occurrence or emergence of one or more adverse events selected from symptom(s) or syndrome(s) of neuropsychiatric medication withdrawal, typically defined based on a Markush grouping of adverse events. By “one or more” is meant that the methods can be practiced without the occurrence or emergence of any of the recited adverse events, that the methods can be practiced without the occurrence of emergence of any particular one of the recited adverse events, or that the methods can be practiced without the occurrence or emergence of any combination of the recited adverse events.

When a drug treatment is said to occur without inducing adverse events, or symptoms or syndromes of medication withdrawal, it will be understood to refer only to physical dependence, adverse events, or symptoms or syndromes of medication withdrawal which in the clinical judgment of a treating physician are significant and resulting from the drug treatment or cessation thereof. In one embodiment, “significant” refers to a moderate or severe adverse event.

The “AIMS” (Abnormal Involuntary Movement Scale) assessment consists of 10 items describing symptoms of dyskinesia. Facial and oral movements (items 1 through 4), extremity movements (items 5 and 6), and trunk movements (item 7) are observed unobtrusively while the subject is at rest; the investigator also makes global judgments on the subject's dyskinesias (items 8 through 10). Each item is rated on a 5-point scale, with a score of zero representing absence of symptoms (for item 10, no awareness), and a score of 4 indicating a severe condition (for item 10, awareness, severe distress). In addition, the AIMS includes 2 yes/no questions that address the subject's dental status. The AIMS Movement Rating Score is defined as the sum of items 1 through 7 (i.e., items 1 through 4, facial and oral movements; items 5 and 6, extremity movements; and item 7, trunk movements). (Guy 1976; Munetz 1988)

As used herein, an “at risk” individual is an individual who is at risk of developing a disorder to be treated or an adverse event to be prevented. This may be shown, for example, by one or more risk factors, which are measurable parameters that correlate with development of a disorder and are known in the art. When a method is said to treat a condition without causing or eliciting an adverse event, it will be understood that the methods can be performed in patients at risk of the adverse event.

The “BARS” (Barnes Akathisia Rating Scale) is a global clinical assessment of akathisia. Barnes 1989. The BARS consists of 4 items related to akathisia: objective observation of akathisia by the investigator, subjective feelings of restlessness by the subject, subjective distress due to akathisia, and global clinical assessment of akathisia. The first 3 items are rated on a 4-point scale, with a score of zero representing absence of symptoms and a score of 3 representing a severe condition. The global clinical evaluation is made on a 6-point scale, with zero representing absence of symptoms and a score of 5 representing severe akathisia. (Barnes 1989; Barnes 2003).

The “BNSS” (Brief Negative Symptom Scale) is a rating scale to measure the current level of severity of negative symptoms in schizophrenia and schizoaffective disorder (Kirkpatrick 2011). The measure is comprised of 13 individual items and 6 subscale scores (blunted affect, alogia, avolition, anhedonia, asociality, and distress). The 6 subscale scores provide a summary score and the 13 individual items provide a composite total score (ranging from 0 to 78). Each of the items are scored on a Likert-type 7 point scale from 0-6, where values of 0 indicates symptom is absent and a value of 6 means the symptom is a severe form. The number of items varies per subscale.

The “CGI-I” (Clinical Global Impressions-Improvement Scale) is a standard 7-point scale (Guy-1976) that requires the clinician to assess how much the subject's overall symptoms have improved or worsened relative to a baseline state.

The “CGI-S” (Clinical Global Impression-Severity Scale) is a standardized, clinician-administered global rating scale that measures disease severity on a 7-point Likert scale. A higher score on the CGI-S represents a higher severity of disease. To perform this assessment, the rater or investigator answers the following question: “Considering your total clinical experience with this particular population, how mentally ill is the patient at this time?” Response choices include: 1=normal, not at all ill; 2=borderline mentally ill; 3=mildly ill; 4=moderately ill; 5=markedly ill; 6=severely ill; and 7=among the most extremely ill patients.

As used herein, a “clinically significant” or “clinically meaningful” improvement can mean an improvement which is both statistically significant and meaningful from a patient's, clinician's, or caregiver's perspective, typically based on a static measure such as CGI-S or a retrospective evaluation of improvement such as the CGI-I, as described generally in various publications of the United States Food and Drug Administration, including FDA 2018, FDA 2019, and FDA 2020. When a treatment or benefit is described herein, it will be understood that the treatment or benefit preferably shows clinically significant efficacy in a population of patients to a degree of statistical significance.

The “C-SSRS” (Columbia Suicide Severity Rating Scale) is a tool designed to systematically assess and track suicidal adverse events (suicidal behavior and suicidal ideation). The strength of this suicide classification system is in its ability to comprehensively identify suicidal events while limiting the over-identification of suicidal behavior. The scale takes approximately 5 minutes to administer (Posner-2007).

As used herein, “delaying” development of a disorder means to defer, hinder, slow, stabilize, and/or postpone development of the disorder. Delay can be of varying lengths of time, depending on the history of the disease and/or the individual being treated.

“DSM-5” refers to the Diagnostic and Statistical Manual of Mental Disorders, Fifth Edition. Terms used herein can be defined by reference to DSM-5 when necessary to give life and meaning to the term. When a person is defined or diagnosed in this document based on DSM-5, it will be understood that the person need not have been diagnosed using the criteria in DSM-5, but that the person would meet the criteria specified in DSM-5 if so diagnosed.

The Healthcare Resource Utilization (HCRU) is the quantification or description of the use of services by persons for the purpose of preventing and curing health problems, promoting maintenance of health and well-being, or obtaining information about one's health status and prognosis. The HCRU includes questions regarding the numbers of physician office visits, emergency room (ER) visits, hospitalizations, and length of hospital stays for any reason and for care during the past 1 month.

The Medication Satisfaction Questionnaire (MSQ) is a single-item, patient-rated questionnaire that requires the subject to use a 7-point, Likert-type scale to rate how satisfied they are with their current antipsychotic medication (antipsychotic medication taken at the time of screening or within 30 days of screening) (Vernon 2010). The subject is asked the following question: “Overall, how satisfied are you with your current medication?” Subjects select 1 of 7 potential responses based on their level of satisfaction, from (1) extremely dissatisfied to (7) extremely satisfied.

The term “neuropsychiatric medication” refers to any medication having neuropsychiatric activity. One class of neuropsychiatric medications is the typical and atypical antipsychotics. Thus, whenever the term “neuropsychiatric medication” is employed herein, the term “typical or atypical antipsychotic” can be substituted therefor.

The “PANSS” (Positive and Negative Syndrome Scale) is an interview-based measure of the severity of psychopathology in adults with psychotic disorders. The measure is comprised of 30 items and 3 subscales: the Positive subscale assesses hallucinations, delusions, and related symptoms; the Negative subscale assesses emotional withdrawal, lack of motivation, and similar symptoms; and the General Psychopathology subscale addresses other symptoms such as anxiety, somatic concern, and disorientation. An anchored Likert scale from 1-7, where values of 2 and above indicate the presence of progressively more severe symptoms, is used to score each item. (Kay-1994, Opler-1992; Perkins-2000).

“Pharmaceutically acceptable” or “physiologically acceptable” refers to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of ulotaront include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions (X) are quite acceptable as synthetic intermediates. Thus, X may be pharmaceutically undesirable anions, such as iodide, oxalate, trifluoromethanesulfonate and the like, when such salts are chemical intermediates.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, which has been approved by or is otherwise acceptable to the United States Food and Drug Administration upon proper qualification for use in humans or domestic animals.

The “PSP” (Personal and Social Performance) Scale is a 100-point single-item rating scale of personal and social functioning (Morosini 2000). The rating is based on the assessment of a patient's functioning in four areas: 1) socially useful activities; 2) personal and social relationships; 3) self-care; and 4) disturbing and aggressive behaviors. Higher scores indicate better functioning. Scores of 0-30 indicate poor functioning; scores of 31-70 indicate varying degrees of difficulty; and scores of 71 100 reflect only mild difficulties at most.

The “PSQI” (Pittsburgh Sleep Quality Index) contains 19 self-rated questions and 5 questions rated by the bed partner or roommate (if one is available). Only self-rated questions are included in the scoring. The 19 self-rated items are combined to form 7 “component” scores, each of which has a range of 0 to 3 points. In all cases, a score of “0” indicates no difficulty, while a score of “3” indicates severe difficulty. The 7 component scores are then added to yield one “global” score, with a range of 0 to 21 points, “0” indicating no difficulty and “21” indicating severe difficulties in all areas.

As used herein, “prevention” or “preventing” or “prevents” refers to a regimen that protects against the onset of the disorder such that the clinical symptoms of the disorder do not develop. Accordingly, “prevention” relates to administration of a therapy to a subject before signs of the diseases are detectable in the subject (for example, administration of a therapy in the absence of a detectable symptom of the disorder). The subject may be an individual at risk of developing the disorder.

The term “a reduced risk of” (or “reduces the risk of”) as used herein means that a subject has a decreased incidence and/or severity of an adverse event or disorder symptom.

The “SAS” (Simpson Angus Scale) consists of a list of 10 symptoms of Parkinsonism (gait, arm dropping, shoulder shaking, elbow rigidity, wrist rigidity, head rotation, glabella tap, tremor, salivation, and akathisia). Simpson 1970. Each item is rated on a 5-point scale, with a score of zero representing absence of symptoms, and a score of 4 representing a severe condition. The SAS Total Score is the sum of the scores for all 10 items.

The term “selecting” refers to the act of choosing from a number or group by fitness or preference. In the context of the current disclosure, ulotaront is selected from a group of generally recognized neuropsychiatric medications, for the treatment of any of the neuropsychiatric conditions described herein.

The “SF 12” (12-Item Short Form Survey) is a 12-item self-reported questionnaire that is a subset of the SF 36 Health Survey. The survey captures physical and mental health. There are 8 subscales including: Physical functioning, Role-physical, Bodily pain, General health, Vitality, Social Functioning, Role emotional, and Mental health. The responses are reported on a 3- or 5-point Likert scale, depending on the question. The SF-12 uses 2 items each to estimate scores for 4 of the 8 health concepts (physical functioning, role-physical, role-emotional, and mental health). Score for the remaining 4 healthy concepts (bodily pain, general health, vitality, and social functioning) are estimated using 1 item each. Physical Component Summary (PCS-12) and Mental Component Summary (MCS-12) are computed using the scores of 12 questions and range from 0 to 100, where a zero score indicates the lowest level of health measured by the scales and 100 indicates the highest level of health.

The “SF-36” (36-Item Short-form Survey) is a self-reported questionnaire with a standard recall period of 4 weeks which measures generic health-related quality of life on 2 broad domains, physical and mental composites, across 8 health domain scales: physical functioning, pain, role physical, general health, vitality/fatigue, social functioning, role emotional, and mental health. The SF-36 uses norm-based scoring to generate scores on a scale of 0 to 100 where lower scores on the physical component summary and mental component summary represent a lower health-related quality of life and a score of 50 references the normative data derived from surveys of representative samples of US general population.

As used herein, the term “significantly” refers to a level of statistical significance. The level of statistical significance can be p<0.1, p<0.05, p<0.01, p<0.005, or p<0.001. Unless otherwise specified, the level of statistical significance when the term “significant,” “significantly,” or other variations of the term are used is p<0.05. When a measurable result or effect is expressed or identified herein, it will be understood that the result or effect is preferably evaluated based upon its statistical significance relative to a baseline such as placebo. In like manner, when a treatment or benefit is described herein, it will be understood that the treatment or benefit preferably shows efficacy in a population of patients to a degree of statistical significance.

The Structured Clinical Interview for DSM-5 Axis I Disorders-Clinical Trials Version (SCID-5-CT) is a modified version of the SCID developed for use in clinical trials. It is a semi-structured interview for the purpose of making a DSM-5 diagnosis (First 2015).

As used herein, “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, “tapering” refers to the period during which a subject is transitioned from the neuropsychiatric medication to ulotaront in the methods of this disclosure and is coterminous with the period of time during which the neuropsychiatric medication and ulotaront are co-administered. The term is also used when referring to ulotaront doses administered during the tapering period (i.e., first ulotaront dose) and neuropsychiatric medication doses administered during the tapering period (i.e., first NM dose). When used to describe a dose, no implication should be drawn from the use of the term tapering as to whether the ulotaront dose is being increased or decreased or staying the same during the tapering period, or whether the neuropsychiatric medication dose is being increased or decreased or staying the same. Furthermore, when two drugs are said to be administered in specified doses during the tapering period, no implication should be drawn as to whether the two drugs in their specified doses are administered at the same time or in what order. The tapering period immediately follows “the first treatment period” (i.e., the period during which only the neuropsychiatric medication is administered).

As used herein, the term “therapeutically effective amount” or “effective amount” refers to an amount that is effective to elicit the desired biological or medical response, including the amount of a compound that, when administered to a subject for treating a disorder, is sufficient to accomplish such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound. A dose given during a “therapeutically effective period” is by definition a “therapeutically effective amount.”

As used herein, the terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed, for example, acute exacerbation of symptoms. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

“Ulotaront” (a/k/a SEP-363856, SEP-363856, SEP-856), as referred to herein for use in the methods of the present disclosure, has the chemical name(S)-(4,5-dihydro-7H-thieno[2,3-c]pyran-7-yl)-N-methylmethanamine (which may be abbreviated as “(S)-TPMA”). Ulotaront has the following structure:

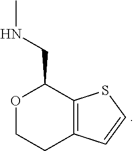

Unless stated otherwise, or unless context requires otherwise, for purposes of this disclosure, the term “ulotaront,” standing alone, includes the free form of ulotaront and also includes its pharmaceutically acceptable salts, hydrates, solvates, amorphous and crystalline forms. When the free form is intended, or any other form or salt is specifically intended, it will be stated as such expressly.

Ulotaront can be used in the methods described herein as the free base or in the form of a pharmaceutically acceptable salt. In preferred embodiments, a hydrochloric acid (HCl) salt of ulotaront is used in the methods described herein. Ulotaront, or a pharmaceutically acceptable salt thereof, including its HCl crystalline forms, can be obtained according to the production methods described in PCT Patent Publication No. WO2011/069063 (U.S. Pat. No. 8,710,245, issued Apr. 29, 2014) or PCT Patent Publication No. WO2019/161238, which are incorporated herein by reference in their entirety and for all purposes, or a method analogous thereto.

Also provided herein are pharmaceutical compositions and dosage forms, comprising ulotaront, or a pharmaceutically acceptable salt thereof, and one or more pharmaceutically acceptable excipients. Compositions and dosage forms provided herein may further comprise one or more additional active ingredients. Ulotaront, or a pharmaceutically acceptable salt thereof, may be administered as part of a pharmaceutical composition as described herein.

### Discussion

In one embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose, and wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the first ulotaront tapering dose is optionally less than the final ulotaront dose, and (ii) the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein the first NM tapering dose is the same as the treatment NM dose, and the first ulotaront tapering dose is optionally less than the final ulotaront dose.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the first NM tapering dose is the same as the treatment NM dose, and (ii) the first ulotaront tapering dose is optionally less than the final ulotaront dose.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication fails to adequately improve the neuropsychiatric disorder; and (ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period, (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication fails adequately to improve the neuropsychiatric disorder; and (ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication affects a target receptor in the subject; and (ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication affects a target receptor in the subject; and (ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

In another embodiment, the disclosure provides a method of switching a human subject being treated with a dose of a neuropsychiatric medication (treatment NM dose) for a neuropsychiatric disorder during a first treatment period to ulotaront comprising: (a) administering to the subject a dose of the neuropsychiatric medication (first NM tapering dose) and a first dose of ulotaront or a pharmaceutically acceptable salt thereof (first ulotaront tapering dose) during a tapering period; and (b) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication produces one or more adverse events in the subject; and (ii) the ulotaront or pharmaceutically acceptable salt thereof does not produce one or more of the adverse events in the subject, optionally wherein the subject takes the neuropsychiatric medication for at least two weeks during the tapering period.

In another embodiment, the disclosure provides a method of treating a neuropsychiatric disorder in a human subject in need thereof comprising: (a) administering to the subject a treatment dose of a neuropsychiatric medication (treatment NM dose) for the disorder during a first treatment period; (b) after the first treatment period, administering a tapering dose of the neuropsychiatric medication (first NM tapering dose) and a first dose of ulotaront or a pharmaceutically acceptable salt thereof (first ulotaront tapering dose) during a tapering period; and (c) after the tapering period, administering a final dose of the ulotaront or pharmaceutically acceptable salt thereof (final ulotaront dose) for a therapeutically effective period of time without administering the neuropsychiatric medication, wherein: (i) the neuropsychiatric medication produces one or more adverse events in the subject; and (ii) the ulotaront or pharmaceutically acceptable salt thereof does not produce one or more of the adverse events in the subject, optionally wherein the subject takes the neuropsychiatric medication for at least two weeks during the tapering period.

In some embodiments of the disclosure, (a) the neuropsychiatric disorder is schizophrenia; (b) the neuropsychiatric medication fails to improve a PANSS score in the subject; and (c) the ulotaront or pharmaceutically acceptable salt thereof improves a PANSS score in the subject.

In other embodiments: (a) the neuropsychiatric medication fails to improve a PANSS score in the subject to a degree of clinical significance; and (b) the ulotaront or pharmaceutically acceptable salt thereof improves a PANSS score in the subject to a degree of clinical significance.

In various embodiments, the subject has a PANSS score ≤80, and optionally ≥30, 34, 38, 42, 46, 50, 54, 58, 62, 66, or 70 immediately prior to commencing ulotaront administration. In further embodiments, the neuropsychiatric medication fails to improve a PANSS score in the subject by ≥10, 14, 18, 22, 26, or 30 points (constituting a lack of clinically significant improvement). In still further embodiments, the subject's PANSS score is improved by ≥14, 18, 22, 26, 30, 34, 38, 42, 46, or 50 points on an absolute basis following commencement of ulotaront administration (constituting a clinically significant improvement). Alternatively, a “clinically significant improvement” can refer to an improvement in PANSS score of at least 20% or at least 30%.

In some embodiments, (a) one or more symptoms of the disorder persist or worsen in the subject during administration of the neuropsychiatric medication; and (b) the ulotaront or pharmaceutically acceptable salt thereof improves one or more of the persistent or worsened symptoms in the subject.

In other embodiments, (a) the neuropsychiatric disorder is schizophrenia; (b) one or more symptoms of the disorder selected from negative symptoms, positive symptoms, disorganized symptoms, hostility symptoms, and depression/anxiety symptoms persist or worsen in the subject during administration of the neuropsychiatric medication; and (c) the ulotaront or pharmaceutically acceptable salt thereof improves one or more of the persistent or worsened symptoms in the subject.

In other embodiments, (a) the neuropsychiatric disorder is schizophrenia; (b) negative symptoms persist or worsen in the subject during administration of the neuropsychiatric medication; and (c) the ulotaront or pharmaceutically acceptable salt thereof improves one or more of the persistent or worsened symptoms in the subject.

In other embodiments, (a) the neuropsychiatric disorder is schizophrenia; (b) positive symptoms persist or worsen in the subject during administration of the neuropsychiatric medication; and (c) the ulotaront or pharmaceutically acceptable salt thereof improves one or more of the persistent or worsened symptoms in the subject.

In other embodiments, (a) the neuropsychiatric disorder is schizophrenia; (b) disorganized symptoms persist or worsen in the subject during administration of the neuropsychiatric medication; and (c) the ulotaront or pharmaceutically acceptable salt thereof improves one or more of the persistent or worsened symptoms in the subject.

In other embodiments, (a) the neuropsychiatric disorder is schizophrenia; (b) hostility symptoms persist or worsen in the subject during administration of the neuropsychiatric medication; and (c) the ulotaront or pharmaceutically acceptable salt thereof improves one or more of the persistent or worsened symptoms in the subject.

In other embodiments, (a) the neuropsychiatric disorder is schizophrenia; (b) depression/anxiety symptoms persist or worsen in the subject during administration of the neuropsychiatric medication; and (c) the ulotaront or pharmaceutically acceptable salt thereof improves one or more of the persistent or worsened symptoms in the subject.

In some embodiments, (a) the neuropsychiatric disorder is schizophrenia; (b) one or more symptoms of the disorder selected from delusions, suspiciousness/persecution, hallucinatory behavior, grandiosity, conceptual disorganization, poor attention, disturbance of volition, lack of judgement and insight, mannerisms and posturing, stereotyped thinking, unusual thought content, preoccupation, difficulty in abstract thinking, disorientation, anxiety, tension, guilt feelings, depression, somatic concern, hostility, uncooperativeness, poor impulse control, excitement, emotional withdrawal, passive/apathetic social withdrawal, active social avoidance, lack of spontaneity and flow of conversation, poor rapport, blunted affect, and motor retardation persist or worsen in the subject during administration of the neuropsychiatric medication; and (c) the ulotaront or pharmaceutically acceptable salt thereof improves one or more of the persistent or worsened symptoms in the subject.

In some embodiments, (a) the neuropsychiatric disorder is depression; (b) one or more symptoms of the disorder selected from apparent sadness, reported sadness, inner tension, reduced sleep, reduced appetite, concentration difficulties, lassitude, inability to feel, pessimistic thoughts, and suicidal thoughts persist or worsen in the subject during administration of the neuropsychiatric medication; and (c) the ulotaront or pharmaceutically acceptable salt thereof improves one or more of the persistent or worsened symptoms in the subject.

In some embodiments, the ulotaront is used to treat anxiety including panic disorder, generalized anxiety disorder, social anxiety disorder, agoraphobia and specific phobia, and symptoms of anxiety selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior. If these symptoms persist or worsen in the subject during administration of the neuropsychiatric medication, switching to the ulotaront improves one or more of the persistent or worsened symptoms in the subject.

In some embodiments, the ulotaront or pharmaceutically acceptable salt thereof reduces nicotine use in the subject.

In some embodiments, (a) nicotine use increases during administration of the neuropsychiatric medication; and (b) the ulotaront or pharmaceutically acceptable salt thereof reduces nicotine use in the subject.

In some embodiments, (a) level of functioning in the subject worsens during administration of the neuropsychiatric medication; and (b) the ulotaront or pharmaceutically acceptable salt thereof improves the level of functioning in the subject.

In some embodiments, the ulotaront or pharmaceutically acceptable salt thereof improves the level of functioning in the subject to a degree of clinical significance.

In some embodiments of the present disclosure, (a) one or more adverse events occur in the subject during administration of the neuropsychiatric medication; and (b) the ulotaront or pharmaceutically acceptable salt thereof improves one or more of the adverse events in the subject. In some embodiments, the adverse events are selected from: (a) abnormal involuntary movements as measured by the AIMS (Abnormal Involuntary Movement Scale); or (b) akathisia, preferably as measured by the BARS (Barnes Akathisia Rating Scale); or (c) suicidality, preferably as measured by the C-SSRS (Columbia-Suicide Severity Rating Scale); or (d) Parkinsonism, preferably as measured by the SAS (Simpson Angus Scale); or (e) sleep quality, preferably as measured by the PSQI (Pittsburgh Sleep Quality Index).

In still further embodiments, the adverse events are selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increased.

In some embodiments, during and/or after the tapering period, the subject has a reduced risk of or does not experience clinically significant withdrawal adverse events selected from cholinergic withdrawal adverse events, dopaminergic withdrawal adverse events (nigrostriatal), dopaminergic withdrawal adverse events (mesolimbic or striatal), serotoninergic withdrawal adverse events, histaminergic withdrawal adverse events, and adrenergic withdrawal adverse events.

In some embodiments, the neuropsychiatric medication is a cholinergic medication and, during and/or after the tapering period, the subject has a reduced risk of or does not experience cholinergic withdrawal adverse events. Put another way, the ulotaront reduces the risk of or prevents one or more cholinergic withdrawal adverse events. The cholinergic withdrawal adverse events can be selected from agitation, insomnia, anxiety or depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion or disorientation, hypothermia, and sweating, among others.

In some embodiments, the neuropsychiatric medication is a dopaminergic medication and, during and/or after the tapering period, the subject has a reduced risk of or does not experience dopaminergic withdrawal adverse events (nigrostriatal). Put another way, the ulotaront reduces the risk of or prevents one or more dopaminergic withdrawal adverse events (nigrostriatal). The dopaminergic withdrawal adverse events (nigrostriatal) can be selected from withdrawal dyskinesia, parkinsonism, neuroleptic malignant syndrome, and akathisia, among others.

In some embodiments, the neuropsychiatric medication is a dopaminergic medication and, during and/or after the tapering period, the subject has a reduced risk of or does not experience dopaminergic withdrawal adverse events (mesolimbic or striatal). Put another way, the ulotaront reduces the risk of or prevents one or more dopaminergic withdrawal adverse events (mesolimbic or striatal). The dopaminergic withdrawal adverse events (mesolimbic or striatal) can be selected from auditory hallucinations, persecutory delusions, and other psychotic symptoms, among others.

In some embodiments, the neuropsychiatric medication is a serotoninergic medication and, during and/or after the tapering period, the subject has a reduced risk of or does not experience serotoninergic withdrawal adverse events. Put another way, the ulotaront reduces the risk of or prevents one or more serotoninergic withdrawal adverse events. The serotoninergic withdrawal adverse events can be selected from flu-like symptoms, sweating or chills, dizziness, light-headedness or tachycardia, paresthesia, electric chock sensations, anxiety, agitation, low mood, insomnia, nightmares, nausea, vomiting, diarrhea, confusion, and decreased concentration, among others.

In some embodiments, the neuropsychiatric medication is a histaminergic medication and, during and/or after the tapering period, the subject has a reduced risk of or does not experience histaminergic withdrawal adverse events. Put another way, the ulotaront reduces the risk of or prevents one or more histaminergic withdrawal adverse events. The histaminergic withdrawal adverse events can be selected from irritability, insomnia, agitation, depressed affect, loss of appetite or nausea, tremulousness, incoordination, and lethargy or amnesia, among others.

In some embodiments, the neuropsychiatric medication is an adrenergic medication and, during and/or after the tapering period, the subject has a reduced risk of or does not experience adrenergic withdrawal adverse events. Put another way, the ulotaront reduces the risk of or prevents one or more adrenergic withdrawal adverse events. The adrenergic withdrawal adverse events can be selected from headache, anxiety or agitation, hypertension, tachycardia, angina, palpitations, risk of myocardial infarction, pre-syncope, tremulousness, and sweating, among others.

In any of the embodiments of the current disclosure, the ulotaront or pharmaceutically acceptable salt thereof produces a clinically significant improvement in the disorder. In further embodiments of the current disclosure, (a) the neuropsychiatric medication fails to produce a clinically significant improvement in the neuropsychiatric disorder; and (b) the ulotaront or pharmaceutically acceptable salt thereof produces a clinically significant improvement in the neuropsychiatric disorder.

In any of the embodiments of the current disclosure, the ulotaront or pharmaceutically acceptable salt thereof produces a clinically significant improvement in one or more symptoms of the disorder. In further embodiments, (a) the neuropsychiatric medication fails to produce a clinically significant improvement in the symptom(s); and (b) the ulotaront or pharmaceutically acceptable salt thereof produces a clinically significant improvement in the symptom(s).

In any of the embodiments of the current disclosure in which the subject experiences one or more adverse events in response to the neuropsychiatric medication, the ulotaront or pharmaceutically acceptable salt thereof produces a clinically significant improvement in the adverse events. In further embodiments, (a) the neuropsychiatric medication produces a clinically significant adverse event profile; and (b) the ulotaront or pharmaceutically acceptable salt thereof produces a clinically significant improvement in the adverse event profile. In further embodiments, (a) the neuropsychiatric medication produces one or more moderate or severe adverse events; and (b) the ulotaront or pharmaceutically acceptable salt thereof improves the one or more moderate or severe adverse events. In still further embodiments, (a) the neuropsychiatric medication produces one or more severe adverse events; and (b) the ulotaront or pharmaceutically acceptable salt thereof improves the one or more severe adverse events.

Various medications can be used as the neuropsychiatric medication. In one embodiment, the neuropsychiatric medication is a D2 receptor antagonist (i.e., conventional antipsychotic). In one embodiment, the neuropsychiatric medication is selected from the group consisting of cholinergic medications, dopaminergic medications, serotoninergic medications, histaminergic medications, and adrenergic medications. In another embodiment, the neuropsychiatric medication is a typical anti-psychotic selected from the group consisting of acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprothepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, and zuclopenthixol. In still further embodiments, the neuropsychiatric medication is an atypical antipsychotic selected from the group consisting of amoxapine, amisulpride, aripiprazole, asenapine, blonanserin, brexpiprazole, cariprazine, carpipramine, clocapramine, clorotepine, clotiapine, clozapine, iloperidone, levosulpiride, lumateperone, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

The tapering period (i.e., the period during which both ulotaront and the neuropsychiatric medication are administered) can last any period of time which the treating physician deems appropriate for reducing adverse effects or improving efficacy when transitioning between the medicines. The tapering period will typically last from 2 to 26 weeks, from 2 to 8 weeks, from 2 to 6 weeks, from 2 to 5 weeks, from 2 to 4 weeks, from 2 to 3 weeks, or 1 week. In some embodiments, the tapering period lasts no more than 6 weeks.

Various tapering regimens also can be employed. In one embodiment, the dose of the neuropsychiatric medication can be tapered downward abruptly or gradually in one or more descending increments. In another embodiment, the dose of the ulotaront or pharmaceutically acceptable salt can be tapered upward abruptly or in one or more ascending increments.

Preferably the ulotaront or pharmaceutically acceptable salt is titrated by administering 50 mg/day for days 1-3 of the first week of the tapering period and 75 mg/day for days 4-7 of the first week of the tapering period, followed by an adjusted dose of 50-100 mg/day (e.g., 50 mg/day, 75 mg/day, or 100 mg/day) for the remainder of the tapering period, which preferably lasts 2-6 weeks in length. The adjusted dose may or may not equal the final ulotaront dose administered during the therapeutically effective period. The first NM tapering dose preferably remains at the treatment NM dose during the first week of the tapering period and is decreased beginning on day 1 of week 2 and eliminated at the end of the tapering period which again is preferably 2-6 weeks after the beginning of the tapering period.

In some embodiments, the first NM tapering dose is the same as the treatment NM dose.

In some embodiments, the tapering period is from 2 to 6 weeks, the first NM tapering dose is the same as the treatment NM dose during at least the first week of the tapering period, and a second tapering dose of the neuropsychiatric medication less than the first NM tapering dose (second NM tapering dose) is administered during the remainder of the tapering period.

In other embodiments, the tapering period is 2-6 weeks, the first NM tapering dose is the same as the treatment NM dose during the first week of the tapering period, and a second tapering dose of the neuropsychiatric medication less than the first NM tapering dose (second NM tapering dose) is administered beginning on week 2 and continuing through the remaining 2-6 weeks of the tapering period.

In some embodiments, the neuropsychiatric medication has a strong binding affinity for muscarinic-cholinergic (M1) and/or histaminergic (H1) receptors, wherein the tapering period lasts 4-6 weeks, the first NM tapering dose is administered for 1 week, and the second NM tapering dose is administered for the remaining 3-5 weeks of the tapering period.

In some embodiments, the neuropsychiatric medication is selected from asenapine, olanzapine, and quetiapine, wherein the tapering period is 4-6 weeks, the first NM tapering dose is administered for 1 week and the second NM tapering dose is administered for the remaining 3-5 weeks.

In some embodiments, the neuropsychiatric medication has a strong binding affinity for dopaminergic receptors, wherein the tapering period lasts 3-5 weeks, the first NM tapering dose is administered for 1 week and the second NM tapering dose is administered for 2-4 weeks.

In some embodiments, the neuropsychiatric medication is selected from lurasidone, paliperidone, risperidone, ziprasidone, haloperidol, and other typical antipsychotics, wherein the tapering period lasts 3-5 weeks, the first NM tapering dose is administered for 1 week and the second NM tapering dose is administered for 2-4 weeks.

In some embodiments, the neuropsychiatric medication has a long half-life, wherein the tapering period lasts 2-4 weeks, the first NM tapering dose is administered for 1 week and the second NM tapering dose is administered for 1-3 weeks.

In some embodiments, the neuropsychiatric medication is selected from aripiprazole, brexpiprazole, and cariprazine, wherein the tapering period lasts 2-4 weeks, the first NM tapering dose is administered for 1 week and the second NM tapering dose is administered for 1-3 weeks.

In some embodiments, the treatment NM dose is at the upper end of the recommended daily dose range, the tapering period lasts 4-6 weeks, the first NM tapering dose is administered for 1 week and the second NM tapering dose is administered for 3-5 weeks.

In some embodiments, the neuropsychiatric medication is a D2 antagonist and the first treatment period exceeds 6 months, 1 year, 2 years, 3 years, or 5 years, the tapering period lasts 4-6 weeks, the first NM tapering dose is administered for 1 week and the second NM tapering dose is administered for 3-5 weeks.

In one embodiment, the first ulotaront tapering dose is less than the final ulotaront dose.

In some embodiments, the first ulotaront tapering dose is less than the final ulotaront dose, further comprising administering a second dose of ulotaront or pharmaceutically acceptable salt thereof during the tapering period (the second ulotaront tapering dose) which is greater than the first ulotaront tapering dose and optionally the same as the final ulotaront dose.

In other embodiments, the tapering period is 2-6 weeks, the first ulotaront tapering dose is administered during days 1-3 of the tapering period, the second ulotaront tapering dose is administered during days 4-7 of the tapering period, and a third ulotaront tapering dose optionally equal to the final ulotaront dose is administered during the remainder of the tapering period.

In further embodiments, the first ulotaront tapering dose is 50 mg/day, the second ulotaront tapering dose is 75 mg/day, and the final ulotaront dose is 50-100 mg/day.

In one embodiment, the treatment NM dose is greater than the first NM tapering dose. In other embodiments, the treatment NM dose is the same as the first NM tapering dose. In other embodiments, the treatment NM dose is the same as the first NM tapering dose, further comprising administering a second tapering dose of the neuropsychiatric medication during the tapering period which is less than the treatment NM dose and the first NM tapering dose. In another embodiment, the first ulotaront tapering dose is the same as the final ulotaront dose. In other embodiments, the first ulotaront tapering dose is less than the final ulotaront dose. In other embodiments, the first ulotaront tapering dose is less than the final ulotaront dose, further comprising administering a second dose of ulotaront or pharmaceutically acceptable salt thereof during the tapering period which is greater than the first ulotaront tapering dose and optionally the same as the final ulotaront dose.

The method can be used to treat many neuropsychiatric disorders treatable by first or second generation antipsychotics, symptoms of those disorders, or side effects associated with the conventional treatment of these disorders. Representative disorders include schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, aggression, delirium, Parkinson's psychosis, excitative psychosis, Tourette's syndrome, organic or NOS psychosis, seizure, agitation, post-traumatic stress disorder, behavior disorder, neurodegenerative disease, Alzheimer's disease, Parkinson's disease, dyskinesias, Huntington's disease, dementia, mood disorder, anxiety, affective disorders (e.g., depression, e.g., major depressive disorder and dysthymia; bipolar disorder, e.g., bipolar depressive disorder; manic disorder; seasonal affective disorder; and attention deficit disorder (ADD) and attention deficit hyperactivity disorder (ADHD)), obsessive-compulsive disorder, vertigo, epilepsy, pain (e.g., neuropathic pain, sensitization accompanying neuropathic pain, and inflammatory pain), fibromyalgia, migraine, cognitive impairment, movement disorder, restless leg syndrome (RLS), multiple sclerosis, sleep disorder, sleep apnea, narcolepsy, excessive daytime sleepiness, jet lag, drowsy side effect of medications, insomnia, substance abuse or dependency (e.g., nicotine, cocaine), addiction, eating disorder, sexual dysfunction, hypertension, emesis, Lesche-Nyhane disease, Wilson's disease, autism, Huntington's chorea, and premenstrual dysphoria.

Given ulotaront's unique behavioral signatures (as reported by Dedic 2019), the human clinical results reported in PCT Patent Publication No. WO 2020/118032, and ulotaront's novel mechanism of action and receptor binding profile, ulotaront can be expected to treat neuropsychiatric disorders with a high degree of safety and efficacy. Thus, in one embodiment, ulotaront is used to improve symptoms in neuropsychiatric disorders selected from psychoses, depression, pain, cognition, mood disorders, and anxiety.

In another embodiment, the neuropsychiatric disorders include schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), psychoaffective disorder, excitative psychosis, organic or NOS psychosis, dyskinesias, mood disorder, anxiety, affective disorders (e.g., depression, e.g., major depressive disorder and dysthymia; bipolar disorder, e.g., bipolar depressive disorder; manic disorder; seasonal affective disorder), pain (e.g., neuropathic pain, sensitization accompanying neuropathic pain, and inflammatory pain), cognitive impairment, movement disorder.

In another embodiment, the treatment is undertaken without inducing a clinically significant occurrence of conditions traditionally associated with neuropsychiatric treatments, including the treatment of such conditions, such as hyperprolactinaemia, blood prolactin abnormal, blood prolactin increased, galactorrhoea, cogwheel rigidity, obesity, metabolic syndrome, dyslipidemia, akathisia, extrapyramidal disorder, restlessness, increased appetite, pancreatitis chronic, weight increased, and nuchal rigidity.

In one embodiment, the neuropsychiatric disorder is selected from schizophrenia, depression, and anxiety. In another embodiment, the neuropsychiatric disorder is schizophrenia. In another embodiment, the neuropsychiatric disorder is bipolar depression (particularly MDD as defined by DSM-5 and more particularly AMDD). In still another embodiment, the neuropsychiatric disorder is anxiety (for example, generalized anxiety disorder (GAD) as defined by DSM-5).

The first ulotaront tapering dose or second ulotaront tapering dose or final ulotaront dose can be described variously as 10-150 mg/day or 25-150 mg/day or 25-100 mg/day or 50-125 mg/day or 50-100 mg/day, administered orally. Alternatively, the first ulotaront tapering dose or second ulotaront tapering dose of final ulotaront dose can be described as 10 mg/day, 15 mg/day, 20 mg/day, 25 mg/day, 50 mg/day, 75 mg/day, 100 mg/day, 125 mg/day, or 150 mg/day, orally administered. In any of the embodiments of the disclosure, the first ulotaront tapering dose or second ulotaront tapering dose or final ulotaront dose can be administered once daily in the fed or fasted state. The ulotaront can also be administered as the hydrochloride salt.

Preferred aspects of the disclosure can be defined based on Embodiments 1 through 74 below:

[Embodiment 1] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

[Embodiment 2] A method of treating a neuropsychiatric disorder in a human subject in need thereof comprising: administering to the subject a treatment dose of a neuropsychiatric medication (treatment NM dose) for the disorder during a first treatment period; after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose.

[Embodiment 3] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose, and wherein the subject takes the neuropsychiatric medication for at least two weeks during the tapering period.

[Embodiment 4] A method of treating a neuropsychiatric disorder in a human subject in need thereof comprising: administering to the subject a treatment dose of a neuropsychiatric medication (treatment NM dose) for the disorder during a first treatment period; after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose, and the subject takes the neuropsychiatric medication for at least two weeks during the tapering period.

[Embodiment 5] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein the first NM tapering dose is the same as the treatment NM dose, and the first ulotaront tapering dose is optionally less than the final ulotaront dose.

[Embodiment 6] A method of treating a neuropsychiatric disorder in a human subject in need thereof comprising: administering to the subject a treatment dose of a neuropsychiatric medication (treatment NM dose) for the disorder during a first treatment period, after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the first NM tapering dose is the same as the treatment NM dose, and the first ulotaront tapering dose is optionally less than the final ulotaront dose.

[Embodiment 7] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication fails to adequately improve the neuropsychiatric disorder; and the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

[Embodiment 8] A method of treating a neuropsychiatric disorder in a human subject in need thereof comprising: administering to the subject a treatment dose of a neuropsychiatric medication (treatment NM dose) for the disorder during a first treatment period; after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication fails adequately to improve the neuropsychiatric disorder; and the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

[Embodiment 9] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication affects a target receptor in the subject; and the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

[Embodiment 10] A method of treating a neuropsychiatric disorder in a human subject in need thereof comprising: administering to the subject a treatment dose of a neuropsychiatric medication (treatment NM dose) for the disorder during a first treatment period; after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication affects a target receptor in the subject; and the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

[Embodiment 11] A method of switching a human subject being treated with a dose of a neuropsychiatric medication (treatment NM dose) for a neuropsychiatric disorder during a first treatment period to ulotaront comprising: administering to the subject a dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication produces one or more adverse events in the subject; and the ulotaront or pharmaceutically acceptable salt thereof does not produce one or more of the adverse events in the subject, optionally wherein the subject takes the neuropsychiatric medication for at least two weeks during the tapering period.

[Embodiment 12] A method of treating a neuropsychiatric disorder in a human subject in need thereof comprising: administering to the subject a treatment dose of a neuropsychiatric medication (treatment NM dose) for the disorder during a first treatment period; after the first treatment period, administering a tapering dose of the neuropsychiatric medication (first NM tapering dose) and a first dose of ulotaront or a pharmaceutically acceptable salt thereof (first ulotaront tapering dose) during a tapering period; and after the tapering period, administering a final dose of the ulotaront or pharmaceutically acceptable salt thereof (final ulotaront dose) for a therapeutically effective period of time without administering the neuropsychiatric medication, wherein: the neuropsychiatric medication produces one or more adverse events in the subject; and the ulotaront or pharmaceutically acceptable salt thereof does not produce one or more of the adverse events in the subject, optionally wherein the subject takes the neuropsychiatric medication for at least two weeks during the tapering period.

[Embodiment 13] The method of embodiment 11 or 12, wherein the ulotaront or pharmaceutically acceptable salt thereof resolves the one or more adverse events.

[Embodiment 14] The method of any of embodiments 1-12, wherein: the neuropsychiatric disorder is schizophrenia; the neuropsychiatric medication fails to improve a PANSS score in the subject; and the ulotaront or pharmaceutically acceptable salt thereof improves a PANSS score in the subject.

[Embodiment 15] The method of any of embodiments 1-12, wherein: the neuropsychiatric disorder is schizophrenia; the neuropsychiatric medication fails to improve a PANSS score in the subject to a degree of clinical significance; and the ulotaront or pharmaceutically acceptable salt thereof improves a PANSS score in the subject to a degree of clinical significance.

[Embodiment 16] The method of any of embodiments 1-12, wherein: the neuropsychiatric disorder is schizophrenia; the subject has a PANSS score ≤80, and optionally ≥30, 34, 38, 42, 46, 50, 54, 58, 62, 66, or 70 immediately prior to commencing ulotaront administration; and the subject's PANSS score is improved by ≥10, 12, 14, 16, or 18 points on an absolute basis.

[Embodiment 17] The method of any of embodiments 1-12, wherein: one or more symptoms of the disorder persist or worsen in the subject during administration of the neuropsychiatric medication; and the ulotaront or pharmaceutically acceptable salt thereof improves one or more of the persistent or worsened symptoms in the subject.

[Embodiment 18] The method of any of embodiments 1-12, wherein: the neuropsychiatric disorder is schizophrenia; one or more symptoms of the disorder selected from negative symptoms, positive symptoms, disorganized symptoms, hostility symptoms, and depression/anxiety symptoms persist or worsen in the subject during administration of the neuropsychiatric medication; and the ulotaront or pharmaceutically acceptable salt thereof improves one or more of the persistent or worsened symptoms in the subject.

[Embodiment 19] The method of any of embodiments 1-12, wherein: the neuropsychiatric disorder is schizophrenia; one or more symptoms of the disorder selected from delusions, suspiciousness/persecution, hallucinatory behavior, grandiosity, conceptual disorganization, poor attention, disturbance of volition, lack of judgement and insight, mannerisms and posturing, stereotyped thinking, unusual thought content, preoccupation, difficulty in abstract thinking, disorientation, anxiety, tension, guilt feelings, depression, somatic concern, hostility, uncooperativeness, poor impulse control, excitement, emotional withdrawal, passive/apathetic social withdrawal, active social avoidance, lack of spontaneity and flow of conversation, poor rapport, blunted affect, and motor retardation persist or worsen in the subject during administration of the neuropsychiatric medication; and the ulotaront or pharmaceutically acceptable salt thereof improves one or more of the persistent or worsened symptoms in the subject.

[Embodiment 20] The method of any of embodiments 1-12, wherein: the neuropsychiatric disorder is depression; one or more symptoms of the disorder selected from apparent sadness, reported sadness, inner tension, reduced sleep, reduced appetite, concentration difficulties, lassitude, inability to feel, pessimistic thoughts, and suicidal thoughts persist or worsen in the subject during administration of the neuropsychiatric medication; and the ulotaront or pharmaceutically acceptable salt thereof improves one or more of the persistent or worsened symptoms in the subject.

[Embodiment 21] The method of any of embodiments 1-12, wherein: the neuropsychiatric disorder is anxiety; one or more symptoms of the disorder selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior persist or worsen in the subject during administration of the neuropsychiatric medication; and the ulotaront or pharmaceutically acceptable salt thereof improves one or more of the persistent or worsened symptoms in the subject.

[Embodiment 22] The method of any of embodiments 1-21, wherein the ulotaront or pharmaceutically acceptable salt thereof reduces nicotine use in the subject.

[Embodiment 23] The method of any of embodiments 1-22, wherein: nicotine use increases during administration of the neuropsychiatric medication; and the ulotaront or pharmaceutically acceptable salt thereof reduces nicotine use in the subject.

[Embodiment 24] The method of any of embodiments 1-23, wherein: level of functioning in the subject worsens during administration of the neuropsychiatric medication; and the ulotaront or pharmaceutically acceptable salt thereof improves the level of functioning in the subject.

[Embodiment 25] The method of any of embodiments 1-24, wherein: level of functioning in the subject worsens during administration of the neuropsychiatric medication; and the ulotaront or pharmaceutically acceptable salt thereof improves the level of functioning in the subject to a degree of clinical significance.

[Embodiment 26] The method of any of embodiments 1-25, wherein: one or more adverse events occur in the subject during administration of the neuropsychiatric medication; and the ulotaront or pharmaceutically acceptable salt thereof improves one or more of the adverse events in the subject.

[Embodiment 27] The method of any of embodiments 1-26, wherein: one or more adverse events occur in the subject during administration of the neuropsychiatric medication; the ulotaront or pharmaceutically acceptable salt thereof improves one or more of the adverse events in the subject; and the adverse events are selected from: (a) abnormal involuntary movements as measured by the AIMS (Abnormal Involuntary Movement Scale); (b) akathisia, preferably as measured by the BARS (Barnes Akathisia Rating Scale); (c) suicidality, preferably as measured by the C-SSRS (Columbia-Suicide Severity Rating Scale); (d) Parkinsonism, preferably as measured by the SAS (Simpson Angus Scale); and (e) sleep quality, preferably as measured by the PSQI (Pittsburgh Sleep Quality Index).

[Embodiment 28] The method of any of embodiments 1-27, wherein: one or more adverse events occur in the subject during administration of the neuropsychiatric medication; the ulotaront or pharmaceutically acceptable salt thereof improves one or more of the adverse events in the subject; and the adverse events are selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increase.

[Embodiment 29] The method of any of embodiments 1-28, wherein during and/or after the tapering period, the subject has a reduced risk of or does not experience clinically significant withdrawal adverse events selected from cholinergic withdrawal adverse events, dopaminergic withdrawal adverse events (nigrostriatal), dopaminergic withdrawal adverse events (mesolimbic or striatal), serotoninergic withdrawal adverse events, histaminergic withdrawal adverse events, and adrenergic withdrawal adverse events.

[Embodiment 30] The method of any of embodiments 1-29, wherein the neuropsychiatric medication is a cholinergic medication and, during and/or after the tapering period, the subject has a reduced risk of or does not experience one or more cholinergic withdrawal adverse events.

[Embodiment 31] The method of any of embodiments 1-29, wherein the neuropsychiatric medication is a cholinergic medication and, during and/or after the tapering period, the subject has a reduced risk of or does not experience cholinergic withdrawal adverse events selected from agitation, insomnia, anxiety, depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion, disorientation, hypothermia, and sweating.

[Embodiment 32] The method of any of embodiments 1-29, wherein the neuropsychiatric medication is a dopaminergic medication and, during and/or after the tapering period, the subject has a reduced risk of or does not experience dopaminergic withdrawal adverse events (nigrostriatal).

[Embodiment 33] The method of any of embodiments 1-29, wherein the neuropsychiatric medication is a dopaminergic medication and, during and/or after the tapering period, the subject has a reduced risk of or does not experience dopaminergic withdrawal adverse events (nigrostriatal) selected from withdrawal dyskinesia, parkinsonism, neuroleptic malignant syndrome, and akathisia.

[Embodiment 34] The method of any of embodiments 1-29, wherein the neuropsychiatric medication is a dopaminergic medication and, during and/or after the tapering period, the subject has a reduced risk of or does not experience dopaminergic withdrawal adverse events (mesolimbic or striatal).

[Embodiment 35] The method of any of embodiments 1-29, wherein the neuropsychiatric medication is a dopaminergic medication and, during and/or after the tapering period, the subject has a reduced risk of or does not experience dopaminergic withdrawal adverse events (mesolimbic or striatal) selected from auditory hallucinations, persecutory delusions, and other psychotic symptoms.

[Embodiment 36] The method of any of embodiments 1-29, wherein the neuropsychiatric medication is a serotoninergic medication and, during and/or after the tapering period, the subject has a reduced risk of or does not experience serotoninergic withdrawal adverse events.

[Embodiment 37] The method of any of embodiments 1-29, wherein the neuropsychiatric medication is a serotoninergic medication and, during and/or after the tapering period, the subject has a reduced risk of or does not experience serotoninergic withdrawal adverse events selected from flu-like symptoms, sweating, chills, dizziness, light-headedness, tachycardia, paresthesia, electric chock sensations, anxiety, agitation, low mood, insomnia, nightmares, nausea, vomiting, diarrhea, confusion, and decreased concentration.

[Embodiment 38] The method of any of embodiments 1-29, wherein the neuropsychiatric medication is a histaminergic medication and, during and/or after the tapering period, the subject has a reduced risk of or does not experience histaminergic withdrawal adverse events.

[Embodiment 39] The method of any of embodiments 1-29, wherein the neuropsychiatric medication is a histaminergic medication and, during and/or after the tapering period, the subject has a reduced risk of or does not experience histaminergic withdrawal adverse events selected from irritability, insomnia, agitation, depressed affect, loss of appetite, nausea, tremulousness, incoordination, lethargy, and amnesia.

[Embodiment 40] The method of any of embodiments 1-29, wherein the neuropsychiatric medication is an adrenergic medication and, during and/or after the tapering period, the subject has a reduced risk of or does not experience adrenergic withdrawal adverse events.

[Embodiment 41] The method of any of embodiments 1-29, wherein the neuropsychiatric medication is an adrenergic medication and, during and/or after the tapering period, the subject has a reduced risk of or does not experience adrenergic withdrawal adverse events selected from headache, anxiety, agitation, hypertension, tachycardia, angina, palpitations, risk of myocardial infarction, pre-syncope, tremulousness, and sweating.

[Embodiment 42] The method of any of embodiments 1-41, wherein the ulotaront or pharmaceutically acceptable salt thereof produces a clinically significant improvement in the disorder.

[Embodiment 43] The method of any of embodiments 1-42, wherein the neuropsychiatric medication fails to improve one or more symptoms of the disorder, and the ulotaront or pharmaceutically acceptable salt thereof produces a clinically significant improvement in the symptoms.

[Embodiment 44] The method of any of embodiments 1-43, wherein the neuropsychiatric medication produces one or more adverse events and the ulotaront or pharmaceutically acceptable salt thereof produces a clinically significant improvement in the adverse events.

[Embodiment 45] The method of any of embodiments 1-44, wherein: the neuropsychiatric medication fails to produce a clinically significant improvement in the neuropsychiatric disorder; and the ulotaront or pharmaceutically acceptable salt thereof produces a clinically significant improvement in the neuropsychiatric disorder.

[Embodiment 46] The method of any of embodiments 1-45, wherein: the neuropsychiatric medication fails to produce a clinically significant improvement in one or more symptom(s) of the disorder; and the ulotaront or pharmaceutically acceptable salt thereof produces a clinically significant improvement in the symptom(s).

[Embodiment 47] The method of any of embodiments 1-46, wherein: the neuropsychiatric medication produces one or more moderate or severe adverse events; and the ulotaront or pharmaceutically acceptable salt thereof improves the one or more moderate or severe adverse events.

[Embodiment 48] The method of any of embodiments 1-47, wherein: the neuropsychiatric medication produces one or more severe adverse events; and the ulotaront or pharmaceutically acceptable salt thereof improves the one or more severe adverse events.

[Embodiment 49] The method of any of embodiments 1-48, wherein the neuropsychiatric medication is selected from the group consisting of cholinergic medications, dopaminergic medications, serotoninergic medications, histaminergic medications, and adrenergic medications.

[Embodiment 50] The method of any of embodiments 1-48, wherein the neuropsychiatric medication is an anti-psychotic selected from the group consisting of acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprothepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, and zuclopenthixol.

[Embodiment 51] The method of any of embodiments 1-48, wherein the neuropsychiatric medication is an antipsychotic selected from the group consisting of amoxapine, amisulpride, aripiprazole, asenapine, blonanserin, brexpiprazole, cariprazine, carpipramine, clocapramine, clorotepine, clotiapine, clozapine, iloperidone, levosulpiride, lumateperone, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

[Embodiment 52] The method of any of embodiments 1-51, wherein the first NM tapering dose is the same as the treatment NM dose.

[Embodiment 53] The method of any of embodiments 1-52, wherein the first NM tapering dose is the same as the treatment NM dose, further comprising administering a second tapering dose of the neuropsychiatric medication during the tapering period (second NM tapering dose) which is less than the treatment NM dose and the first NM tapering dose.

[Embodiment 54] The method of any of embodiments 1-53, wherein the tapering period is 2-6 weeks, the first NM tapering dose is the same as the treatment NM dose during the first week of the tapering period, and a second tapering dose of the neuropsychiatric medication less than the first NM tapering dose (second NM tapering dose) is administered during the remainder of the tapering period.

[Embodiment 55] The method of any of embodiments 53-54, wherein the neuropsychiatric medication has a strong binding affinity for muscarinic-cholinergic (M1) and/or histaminergic (H1) receptors, wherein the first NM tapering dose is administered for 1 week and the second NM tapering dose is administered for 3-5 weeks.

[Embodiment 56] The method of any of embodiments 53-54, wherein the neuropsychiatric medication is selected from asenapine, olanzapine, and quetiapine, wherein the first NM tapering dose is administered for 1 week and the second NM tapering dose is administered for 3-5 weeks.

[Embodiment 57] The method of any of embodiments 53-54, wherein the neuropsychiatric medication has a strong binding affinity for dopaminergic receptors, wherein the first NM tapering dose is administered for 1 week and the second NM tapering dose is administered for 2-4 weeks.

[Embodiment 58] The method of any of embodiments 53-54, wherein the neuropsychiatric medication is selected from lurasidone, paliperidone, risperidone, ziprasidone, haloperidol, and other typical antipsychotics, wherein the first NM tapering dose is administered for 1 week and the second NM tapering dose is administered for 2-4 weeks.

[Embodiment 59] The method of any of embodiments 53-54, wherein the neuropsychiatric medication has a long half-life, wherein the first NM tapering dose is administered for 1 week and the second NM tapering dose is administered for 1-3 weeks.

[Embodiment 60] The method of any of embodiments 53-54, wherein the neuropsychiatric medication is selected from aripiprazole, brexpiprazole, and cariprazine, wherein the first NM tapering dose is administered for 1 week and the second NM tapering dose is administered for 1-3 weeks.

[Embodiment 61] The method of any of embodiments 53-54, wherein the treatment NM dose is at the upper end of the recommended daily dose range, wherein the first NM tapering dose is administered for 1 week and the second NM tapering dose is administered for 3-5 weeks.

[Embodiment 62] The method of any of embodiments 53-54, wherein the neuropsychiatric medication is a D2 antagonist and the first treatment period exceeds 6 months, 1 year, 2 years, 3 years, or 5 years, wherein the first NM tapering dose is administered for 1 week and the second NM tapering dose is administered for 3-5 weeks.

[Embodiment 63] The method of any of embodiments 1-62, wherein the first ulotaront tapering dose is less than the final ulotaront dose.

[Embodiment 64] The method of any of embodiments 1-63, wherein the first ulotaront tapering dose is less than the final ulotaront dose, further comprising administering a second dose of ulotaront or pharmaceutically acceptable salt thereof during the tapering period (the second ulotaront tapering dose) which is greater than the first ulotaront tapering dose and optionally the same as the final ulotaront dose.

[Embodiment 65] The method of embodiment 64, wherein the tapering period is 2-6 weeks, the first ulotaront tapering dose is administered during days 1-3 of the tapering period, the second ulotaront tapering dose is administered during days 4-7 of the tapering period, and a third ulotaront tapering dose which is optionally the same as the final ulotaront dose is administered during the remainder of the tapering period.

[Embodiment 66] The method of any of embodiments 64-65, wherein the first ulotaront tapering dose is 50 mg/day, the second ulotaront tapering dose is 75 mg/day, and the final ulotaront dose is 50-100 mg/day.

[Embodiment 67] The method of any of embodiments 1-66, wherein the neuropsychiatric disorder is selected from the group consisting of schizophrenia, depression, and anxiety.

[Embodiment 68] The method of any of embodiments 1-66, wherein the neuropsychiatric disorder is schizophrenia.

[Embodiment 69] The method of any of embodiments 1-66, wherein the neuropsychiatric disorder is MDD.

[Embodiment 70] The method of any of embodiments 1-66, wherein the neuropsychiatric disorder is GAD.

[Embodiment 71] The method of any of embodiments 1-70, wherein the first tapering ulotaront dose and the final ulotaront dose are independently selected from 10-150 mg/day or 25-100 mg/day or 50-125 mg/day or 50-100 mg/day or 50-75 mg/day, orally administered.

[Embodiment 72] The method of any of embodiments 1-70, wherein the first tapering ulotaront dose and the final ulotaront dose are independently selected from 10 mg/day, 15 mg/day, 20 mg/day, 25 mg/day, 50 mg/day, 75 mg/day, 100 mg/day, 125 mg/day, or 150 mg/day, orally administered.

[Embodiment 73] The method of any one of embodiments 1-72, wherein the first tapering ulotaront dose and the final ulotaront dose are administered once daily in the fed or fasted state.

[Embodiment 74] The method of any one of embodiments 1-73, wherein the ulotaront is administered as the hydrochloride salt.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the disclosure and are not intended to limit the scope of what the inventors regard as their disclosure.

### Example 1: Effect of SEP-363856 on the Body Weight of Female Sprague Dawley Rats Previously Treated with Olanzapine

SEP-363856 was investigated to explore the effects of administration of SEP-363856 on the body weight and metabolic parameters of rats previously treated with olanzapine, including exploring whether administration of the test compound restores body weight to or beyond control levels. Animals received either vehicle or olanzapine once daily for a period of 7 days. An increase in body weight of the animals receiving olanzapine compared to vehicle-treated animals was shown. Subsequently, from Day 8 onwards, the olanzapine-treated animals either continued to receive olanzapine, were switched to vehicle, or were switched to SEP-363856 (0.3, 1 and 3 mg/kg, po).

### Materials and Methods

### Animals:

Seventy-four (2 spares) female Sprague Dawley rats (weight range 200-250 g) were ordered from Charles River, Margate, Kent. Rats were singly housed in cages with sawdust bedding and enrichment at an ambient temperature of 21±2 °C. Upon arrival (Thursday), rats were weighed and given wet mash to aid recovery from transport. Rats were weighed the following day and the Monday of the following week. Animals were maintained on a reverse-phase light-dark cycle (lights off for 8 h from 10:00-18:00 h) during which time the room was illuminated by red light. Rats were weighed on the Friday and Monday of the following week. Relative humidity was typically 55±15% with prolonged periods below 40% RH or above 70% RH avoided as detailed in the UK Code of Practice. All animals had free access to a high-fat powdered diet (VRF1 plus 20% lard) and all rats had access to filtered water at all times. The diets were contained in glass feeding jars with aluminum lids (Solmedia Laboratory Suppliers, Romford, Essex). Each lid had a 3-4 cm hole cut in it to allow access to the food. Animals were accustomed to these conditions for at least two weeks before experimentation.

### Experimental Procedures:

Animals had a 3-day baseline run-in period during which time all rats were dosed once a day with vehicle. Dosing was timed to start at approximately 08:45 so that the mid-point was approximately the time of lights out. Animals, food and water were weighed at the time of dosing. Towards the end of this baseline period, animals were weighed (to the nearest 0.1 g using an electronic top-pan balance) and allocated into 2 weight-matched treatment groups by a Statistician. Subsequently, rats were dosed once daily as follows: Treatment 1 (po) Days 1-4 or 1-7: Group A—Vehicle (3 mL/kg) (n=12); Groups B-F—Olanzapine (3 mg/kg) (n=62).

Olanzapine (3 mg/kg po) has previously been shown in-house to increase body weight by 4-6% in this model. Dosing began at approximately 08:45 h each day (0 h), i.e., so the mid-point of dosing was approximately at the time of lights out. This strategy was taken to maximize the impact of the drugs on food intake. Dosing continued for 7 consecutive days and rats were weighed (to the nearest 0.1 g) every day at 0 h. Prior to and at the completion of each dosing session animals were examined and any overt behavioral/physiological effects or other relevant observations regarding the condition of the animals was recorded manually. From Day 4 onwards, body weight data was analyzed to assess whether a statistically significant increase in weight was evident with olanzapine compared to vehicle-treated controls fed the high-fat diet. When this reached statistical significance, rats in groups B-F were allocated further by a Statistician on the basis of available body weight as follows: Treatment 2: Group A—Vehicle (aqueous methyl cellulose, 1%, 3 mL/kg po) (n=12); Group B—Olanzapine (3 mg/kg po) (n=12); Group C—SEP-363856 (0.3 mg/kg po) (n=12); Group D—SEP-363856 (1 mg/kg po) (n=12); Group E—SEP-363856 (3 mg/kg po) (n=12); Group F—Vehicle (water, 3 mL/kg) (n=12). Subsequently, from Day 8 onwards, animals either continued to receive olanzapine, or were switched to vehicle or SEP-363856 (0.3, 1 and 3 mg/kg, po). Body intake and food intake were assessed daily.

Dosing began at approximately 08:45 h each day (0 h), i.e., the mid-point of dosing was approximately at the time of lights out. Body weight, food and water intake was recorded daily at the time of dosing. All rats were observed before and after dosing and comments on condition were noted as appropriate. On the afternoon of Day 14 (approx. 16:00) animals were fasted to a timed schedule. The experiment ended the following morning on Day 15. On the morning of Day 15, final readings were undertaken 16 h post-fast (i.e., body weight measurements). Animals were then dosed (as described). Four hours later, two blood samples (20 μL and 1 mL) were taken from the lateral tail vein into tubes containing lithium heparin. The 20 μL sample was stored frozen (whole blood) prior to determination of HbA1c (Roche Tina-quant A2c Gen 3, Cobas C111). The larger blood sample was centrifuged at 2,400 g for 5 min at 4 °C to produce five aliquots of plasma which were stored frozen for optional plasma analysis at Sygnature Discovery Ltd (glucose, insulin, triglycerides, cholesterol (HDL, LDL, total) and NEFA). Animals were then terminated by a Schedule 1 method (exposure to an increasing concentration of CO₂ with death confirmed by cervical dislocation). A terminal blood sample (circa 4 mL) was taken into EDTA-coated tube and a single plasma aliquot (approximately 1 mL) was stored frozen (approx. −80 °C) in a clean aliquot tube. The liver was dissected and the caudate lobe removed. Both the caudate lobe and the remaining liver were weighed and stored frozen separately (on dry ice and then transferred to a freezer at approx. −80 °C). The caudate lobe underwent further analysis of liver triglycerides. Carcasses were stored frozen (approx. −20 °C) for potential body composition analysis. Metabolic parameters, including fasting glucose and insulin levels, liver triglycerides and body fat composition, were determined at the end of the study.

### Results

In line with previous publications, olanzapine (3 mg/kg/day, po) significantly increased body weight (Table 2.2.1) and food intake (Table 2.2.2) compared to vehicle treated rats. Initial effects were observed as early as 2-3 days following dosing. On day 8, animals either continued to receive olanzapine or were switched to vehicle or SEP-363856 treatment. The switch to SEP-363856 treatment reversed olanzapine-induced weight gain and food intake at all doses tested (0.3, 1 and 3 mg/kg, po). Importantly, a more rapid reversal of olanzapine-induced weight gain was seen in rats switched to SEP-363856 treatment compared to vehicle. In addition, body composition analysis revealed a significant reduction in percent fat in animals switched to SEP-363856 treatment compared to rats that continuously received olanzapine (Table 2.2.3). Liver triglycerides levels were decreased with SEP-363856 but the reduction did not reach statistical significance (Table 2.2.4). These preliminary results suggest that SEP-363856 reduces weight gain associated with prior antipsychotic drug treatment. Note, SEP-363856 is referred to as Compound 1 in the Tables.

TABLE 2.2.1
Changes in body weights (g)

| | | | | | vs Vehicle (A) | | vs Vehicle (F) | | vs Olanzapine (B) | |
|---|---|---|---|---|---|---|---|---|---|---|
| Time | Treatment | n | Mean | SEM | Difference | p | Difference | p | Difference | p |
| Week 1 | Vehicle po | 12 | 12.41 | 2.04 | | | | | | |
| (Day 1-8) | Olanzapine 3 mg/kg po | 61 | 25.48 | 0.93 | 13.07 | <0.001*** | | | | |
| Week 2 | Vehicle (aqueous methyl | 12 | 9.15 | 2.00 | | | | | | |
| | cellulose) po | | | | | | | | | |
| (Day 8-14) | Olanzapine-3 mg/kg po | 12 | 16.70 | 1.91 | 7.55 | 0.006** | | | | |
| | Compound 1-0.3 mg/kg po | 12 | −1.19 | 1.91 | −10.34 | <0.001*** | −5.85 | 0.025* | −17.90 | <0.001*** |
| | Compound 1-1 mg/kg po | 12 | −6.37 | 1.91 | −15.52 | <0.001*** | −11.03 | <0.001*** | −23.08 | <0.001*** |
| | Compound 1-3 mg/kg po | 12 | −5.31 | 1.91 | −14.46 | <0.001*** | −9.97 | <0.001*** | −22.02 | <0.001*** |
| | Vehicle (water) po | 13 | 4.65 | 1.83 | −4.50 | 0.098 | | | −12.05 | <0.001*** |

Means are estimated from the mixed effects model.
Standard errors of the mean (SEM) are calculated from the residuals of the statistical model.
Compound 1 was compared to Vehicle (A) and Olanzapine (B) by separate Dunnett's tests and Vehicle (F) by Williams' test.
Other comparisons by multiple t test.
* p < 0.05. ** p < 0.01, *** p < 0.001.

TABLE 2.2.2
Averages of daily food intakes (g/day)

| | | | | | vs Vehicle (A) | | vs Vehicle (F) | | vs Olanzapine (B) | |
|---|---|---|---|---|---|---|---|---|---|---|
| Time | Treatment | n | Mean | SEM | Difference | p | Difference | p | Difference | p |
| Week 1 | Vehicle (aqueous methyl | 12 | 15.31 | 0.35 | | | | | | |
| | cellulose) po | | | | | | | | | |
| (Day 1-7) | Olanzapine 3 mg/kg po | 61 | 17.21 | 0.18 | 1.90 | <0.001*** | | | | |
| Week 2 | Vehicle (aqueous methyl | 12 | 15.13 | 0.36 | | | | | | |
| | cellulose) po | | | | | | | | | |
| (Day 8-13) | Olanzapine 3 mg/kg po | 12 | 16.73 | 0.32 | 1.60 | <0.001*** | | | | |
| | Compound 1-0.3 mg/kg po | 12 | 12.98 | 0.32 | −2.14 | <0.001*** | −1.89 | <0.001*** | −3.75 | <0.001*** |
| | Compound 1-1 mg/kg po | 12 | 12.17 | 0.32 | −2.95 | <0.001*** | −2.70 | <0.001*** | −4.56 | <0.001*** |
| | Compound 1-3 mg/kg po | 12 | 12.41 | 0.32 | −2.71 | <0.001*** | −2.46 | <0.001*** | −4.31 | <0.001*** |
| | Vehicle (water) po | 13 | 14.67 | 0.31 | −0.26 | 0.573 | | | −1.86 | <0.001*** |

Means are estimated from the mixed effects model.
Standard errors of the mean (SEM) are calculated from the residuals of the statistical model.
Compound 1 was compared to Vehicle (A) and Olanzapine (B) by separate Dannett's tests and Vehicle (F) by Williams' test.
Other comparisons by multiple t test.
**p < 0.01,
***p < 0.001.

TABLE 2.2.3
Body composition data

| | | | | vs Vehicle (A) | | | vs Vehicle (F) | | vs Olanzapine (B) | |
|---|---|---|---|---|---|---|---|---|---|---|
| Treatment | n | Mean | SEM | Difference | % change | p | Difference | p | Difference | p |
| Water in carcass (%) | | | | | | | | | | |
| Vehicle (aqueous methyl | 12 | 62.6 | 0.5 | | | | | | | |
| cellulose) po | | | | | | | | | | |
| Olanzapine-3 mg/kg po | 12 | 60.4 | 0.4 | −2.2 | −3.6 | 0.006** | | | | |
| Compound 1-0.3 mg/kg po | 12 | 63.1 | 0.5 | 0.6 | 0.9 | 0.816 | 0.6 | 0.585 | 2.8 | 0.002** |
| Compound 1-1 mg/kg po | 12 | 63.0 | 0.5 | 0.4 | 0.6 | 0.934 | 0.5 | 0.585 | 2.6 | 0.004** |
| Compound 1-3 mg/kg po | 12 | 63.1 | 0.7 | 0.5 | 0.8 | 0.866 | 0.6 | 0.585 | 2.7 | 0.003** |
| Vehicle (water) po | 13 | 62.5 | 0.6 | −0.1 | −0.1 | 0.913 | | | 2.1 | 0.007** |
| Water per rat (g) | | | | | | | | | | |
| Vehicle (aqueous methyl | 12 | 163.4 | 1.4 | | | | | | | |
| cellulose) po | | | | | | | | | | |
| Olanzapine-3 mg/kg po | 12 | 163.9 | 1.6 | 0.6 | 0.3 | 0.835 | | | | |
| Compound 1-0.3 mg/kg po | 12 | 165.2 | 2.2 | 1.9 | 1.1 | 0.835 | −2.0 | 0.469 | 1.3 | 0.934 |
| Compound 1-1 mg/kg po | 12 | 160.8 | 1.7 | −2.6 | −1.6 | 0.668 | −6.4 | 0.023* | −3.2 | 0.524 |
| Compound 1-3 mg/kg po | 12 | 160.5 | 2.3 | −2.9 | −1.8 | 0.595 | −6.7 | 0.018* | −3.4 | 0.456 |
| Vehicle (water) po | 13 | 167.2 | 2.1 | 3.8 | 2.3 | 0.159 | | | 3.3 | 0.229 |
| Fat in carcass (%) | | | | | | | | | | |
| Vehicle (aqueous methyl | 12 | 13.4 | 0.6 | | | | | | | |
| cellulose) po | | | | | | | | | | |
| Olanzapine-3 mg/kg po | 12 | 15.8 | 0.6 | 2.4 | 18.3 | 0.016* | | | | |
| Compound 1-0.3 mg/kg po | 12 | 12.5 | 0.6 | −0.9 | −6.5 | 0.709 | −1.0 | 0.399 | −3.3 | 0.004** |
| Compound 1-1 mg/kg po | 12 | 12.6 | 0.6 | −0.8 | −5.7 | 0.784 | −0.9 | 0.399 | −3.2 | 0.005** |
| Compound 1-3 mg/kg po | 12 | 12.4 | 0.7 | −0.9 | −7.0 | 0.663 | −1.1 | 0.350 | −3.4 | 0.003** |
| Vehicle (water) po | 13 | 13.5 | 0.7 | 0.1 | 1.0 | 0.893 | | | −2.3 | 0.020* |
| Fat per rat (g) | | | | | | | | | | |
| Vehicle (aqueous methyl | 12 | 35.3 | 1.6 | | | | | | | |
| cellulose) po | | | | | | | | | | |
| Olanzapine-3 mg/kg po | 12 | 43.3 | 1.7 | 8.0 | 22.7 | 0.007** | | | | |
| Compound 1-0.3 mg/kg po | 12 | 32.7 | 2.1 | −2.6 | −7.4 | 0.698 | −3.7 | 0.201 | −10.6 | 0.001** |
| Compound 1-1 mg/kg po | 12 | 32.3 | 1.6 | −2.9 | −8.3 | 0.623 | −4.0 | 0.195 | −10.9 | 0.001** |
| Compound 1-3 mg/kg po | 12 | 32.0 | 1.9 | −3.3 | −9.4 | 0.535 | −4.4 | 0.161 | −11.3 | <0.001*** |
| Vehicle (water) po | 13 | 36.3 | 1.9 | 1.1 | 3.1 | 0.706 | | | −6.9 | 0.018* |
| Protein in carcass (%) | | | | | | | | | | |
| Vehicle (aqueous methyl | 12 | 20.73 | 0.20 | | | | | | | |
| cellulose) po | | | | | | | | | | |
| Olanzapine-3 mg/kg po | 12 | 20.24 | 0.17 | −0.49 | −2.4 | 0.150 | | | | |
| Compound 1-0.3 mg/kg po | 12 | 20.90 | 0.23 | 0.17 | 0.8 | 0.924 | 0.36 | 0.273 | 0.66 | 0.135 |
| Compound 1-1 mg/kg po | 12 | 21.00 | 0.17 | 0.27 | 1.3 | 0.756 | 0.47 | 0.189 | 0.76 | 0.069 |
| Compound 1-3 mg/kg po | 12 | 21.18 | 0.22 | 0.45 | 2.2 | 0.403 | 0.65 | 0.065 | 0.94 | 0.019* |
| Vehicle (water) po | 13 | 20.53 | 0.20 | −0.20 | −0.9 | 0.554 | | | 0.29 | 0.377 |
| Protein per rat (g) | | | | | | | | | | |
| Vehicle (aqueous methyl | 12 | 54.1 | 0.6 | | | | | | | |
| cellulose) po | | | | | | | | | | |
| Olanzapine-3 mg/kg po | 12 | 55.0 | 0.4 | 0.9 | 1.6 | 0.321 | | | | |
| Compound 1-0.3 mg/kg po | 12 | 54.7 | 0.7 | 0.6 | 1.1 | 0.841 | −0.4 | 0.609 | −0.3 | 0.976 |
| Compound 1-1 mg/kg po | 12 | 53.7 | 0.5 | −0.4 | −0.8 | 0.921 | −1.5 | 0.117 | −1.3 | 0.309 |
| Compound 1-3 mg/kg po | 12 | 53.7 | 0.7 | −0.4 | −0.7 | 0.938 | −1.4 | 0.117 | −1.3 | 0.333 |
| Vehicle (water) po | 13 | 55.1 | 0.6 | 1.0 | 1.9 | 0.235 | | | 0.2 | 0.858 |
| Final carcass weight (g) | | | | | | | | | | |
| Vehicle (aqueous methyl | 12 | 261.3 | 1.3 | | | | | | | |
| cellulose) po | | | | | | | | | | |
| Olanzapine-3 mg/kg po | 12 | 271.9 | 2.3 | 10.6 | 4.1 | 0.006** | | | | |
| Compound 1-0.3 mg/kg po | 12 | 261.4 | 4.1 | 0.1 | 0.0 | >0.999 | −6.7 | 0.073 | −10.5 | 0.017* |
| Compound 1-1 mg/kg po | 12 | 255.4 | 2.0 | −5.9 | −2.2 | 0.280 | −12.7 | 0.001** | −16.5 | <0.001*** |
| Compound 1-3 mg/kg po | 12 | 255.3 | 2.1 | −6.0 | −2.3 | 0.264 | −12.8 | <0.001*** | −16.6 | <0.001*** |
| Vehicle (water) po | 13 | 268.1 | 2.8 | 6.8 | 2.6 | 0.068 | | | −3.8 | 0.298 |

Means are adjusted for Day 1 body weight differences between the treatment groups.
Standard errors of the mean (SEM) are calculated from the residuals of the statistical model.
Compound 1 was compared to Vehicle and Olanzapine by separate Dunnett's tests and to Olanzapine/Vehicle by Williams' test.
Other comparisons by multiple t test.
*p < 0.05,
**p < 0.01,
***p < 0.001.

TABLE 2.2.4
Liver triglycerides data

| | | | | vs Vehicle (A) | | vs Vehicle (F) | | vs Olanzapine (B) | |
|---|---|---|---|---|---|---|---|---|---|
| Treatment | n | Mean | SEM | % | p | % | p | % | p |
| Triglycerides (μmol/g) | | | | | | | | | |
| Vehicle (aqueous methyl cellulose) | 12 | 37.1 | 8.2 | | | | | | |
| po | | | | | | | | | |
| Olanzapine-3 mg/kg po | 12 | 37.1 | 8.2 | 100.0 | >0.999 | | | | |
| Compound 1-0.3 mg/kg po | 12 | 26.5 | 6.8 | 71.3 | 0.569 | 84.0 | 0.572 | 71.3 | 0.569 |
| Compound 1-1 mg/kg po | 12 | 20.3 | 3.8 | 54.7 | 0.143 | 64.5 | 0.538 | 54.7 | 0.143 |
| Compound 1-3 mg/kg po | 12 | 29.7 | 6.9 | 80.0 | 0.816 | 94.3 | 0.538 | 80.0 | 0.816 |
| Vehicle (water) po | 13 | 31.5 | 5.4 | 84.8 | 0.593 | | | 84.8 | 0.593 |
| Triglycerides (μmol total) | | | | | | | | | |
| Vehicle (aqueous methyl cellulose) | 12 | 346 | 76 | | | | | | |
| po | | | | | | | | | |
| Olanzapine-3 mg/kg po | 12 | 348 | 79 | 100.4 | 0.989 | | | | |
| Compound 1.-0.3 mg/kg po | 12 | 242 | 64 | 70.0 | 0.538 | 82.8 | 0.547 | 69.7 | 0.529 |
| Compound 1-1 mg/kg po | 12 | 175 | 34 | 50.5 | 0.088 | 59.8 | 0.404 | 50.3 | 0.086 |
| Compound 1.-3 mg/kg po | 12 | 261 | 59 | 75.4 | 0.702 | 89.3 | 0.404 | 75.1 | 0.693 |
| Vehicle (water) po | 13 | 293 | 52 | 84.5 | 0.588 | | | 84.1 | 0.579 |

Means are back transformed and adjusted for termination order and body weight differences between the treatment groups on Day 1.
Standard errors of the mean (SEM) are calculated from the residuals of the statistical model.
Compound 1 was compared to Vehicle (A) and Olanzapine (B) by separate Dunnett's tests and Vehicle (F) by Williams' test.
Other comparisons by the multiple t test.

### Example 2. An 8-Week, Open-Label Study Evaluating the Effectiveness, Safety and Tolerability of SEP-363856 in Subjects with Schizophrenia Switched from Typical or Atypical Antipsychotic Agents

This is an 8-week, open label study evaluating the effectiveness, safety, and tolerability of SEP-363856 in subjects with schizophrenia switched from typical or atypical antipsychotic agents. The 8-week, outpatient, single-group, open-label design with flexible dosing and flexible switch duration was chosen to approximate a usual care setting for stable outpatients with schizophrenia who are in need of a switch from a previous antipsychotic. There is a screening period to confirm diagnosis and ensure enrollment criteria are met. Subjects who satisfy enrollment criteria are maintained on an antipsychotic treatment and enter an 8-week switch period where SEP-363856 is introduced during the first week (i.e., start at 50 mg/day for Days 1-3, followed by 75 mg/day for Days 4-7). The prior antipsychotic is not adjusted during the first week of the study while Investigators are introducing SEP-363856.

Beginning on Day 8, Investigators have the option to down-taper the previous antipsychotic by the end of Week 2, 3, 4, 5, or 6. The variable duration for down-tapering of the prior antipsychotic is intended to approximate a real-world clinical environment whereby the Investigator considers the needs of the individual patient as well as clinically relevant issues that could influence the switch outcome, such as reason for switch, type of prior antipsychotic, dosage, etc. All study subjects who complete the 8-week switch period experience a minimum of 2 weeks on SEP-363856 monotherapy. Study completers are eligible to roll over into the 24-week open label extension study described in Example 3 for purposes of evaluating longer-term safety and tolerability, as well as effectiveness, of SEP-363856 in subjects switched from typical or atypical antipsychotic agents.

### Objectives

### Primary Objective:

To evaluate the safety and effectiveness of switching clinically stable outpatients with schizophrenia from a typical or atypical antipsychotic to SEP-363856 over a period of 8 weeks as assessed by the percentage of subjects who discontinue from the study due to an adverse event or lack of efficacy

### Secondary Objective:

To evaluate the safety and effectiveness of switching clinically stable outpatients with schizophrenia from a typical or atypical antipsychotic to SEP-363856 over a period of 8 weeks as assessed by the percentage of subjects who discontinue for any reason (i.e., all causes of discontinuation)
Other Objectives (1): To evaluate the tolerability and safety of switching clinically stable outpatients with schizophrenia from a typical or atypical antipsychotic to SEP-363856 over a period of 8 weeks using: Adverse event reports, Clinical laboratory tests, Vital sign measurements, 12-lead electrocardiograms (ECG), Columbia-Suicide Severity Rating Scale (C-SSRS), Barnes Akathisia Rating Scale (BARS), Abnormal Involuntary Movement Scale (AIMS), and Simpson-Angus Scale (SAS)
Other Objectives (2): To evaluate the effectiveness of SEP-363856 using: Positive and Negative Syndrome Scale (PANSS), Clinical Global Impression-Severity (CGI-S) scale, Clinical Global Impression-Improvement (CGI-I) scale, and Brief Negative Symptom Scale (BNSS)
Other Objectives (3): To evaluate the effects of SEP-363856 on health-related quality of life as measured by the Short Form Health Survey (SF 12)
Other Objectives (4): To evaluate the effects of SEP-363856 on functional capacity as measured by the Personal and Social Performance Scale (PSP)
Other Objectives (5): To evaluate medication satisfaction as measured by the Medication Satisfaction Questionnaire (MSQ)
Other Objectives (6): To evaluate sleep as measured by the Pittsburgh Sleep Quality Index (PSQI)
Other Objectives (7): To evaluate the impact of SEP-363856 on healthcare resource utilization (HCRU)

### Endpoints

### Primary Endpoint:

Percentage of subjects who discontinue for clinical reasons (i.e., discontinue due to an adverse event [AE] or lack of efficacy)

### Secondary Endpoint:

Percentage of subjects who discontinue for any reason (i.e., all causes for discontinuation)

### Other Endpoints:

The incidence of overall AEs, serious adverse events (SAEs), and AEs leading to discontinuation
Observed values and changes from Baseline in clinical laboratory tests (hematology, chemistry and urinalysis)
Observed values and changes from Baseline in vital signs (including body weight, body mass index [BMI], waist circumference, temperature, blood pressure [supine and standing], heart rate [supine and standing] and respiratory rate)·
Observed values and changes from Baseline in 12-lead ECG parameters
Frequency of subjects with suicidal ideation and suicidal behavior based on the C-SSRS
Changes from Baseline in BARS, AIMS and SAS scores
Changes from Baseline to Week 8 in: (i) PANSS total score and subscale scores (positive, negative, and general psychopathology), (ii) PANSS Marder Factor (five-factor) scores (positive, disorganized, negative, hostility, and depression/anxiety), and Uncorrelated PANSS Score Matrix (UPSM), (iii) CGI-S score, (iv) CGI-I score, (v) BNSS total score, (vi) SF-12 scores, (vii) PSP total score, (viii) MSQ score, (ix) PSQI global score
HCRU (including numbers of physician office visits, emergency room [ER] visits and hospitalizations, length of hospital stays, employment status and average number of hours caregiver(s) spends helping subjects per week.)
Nicotine use at baseline and Week 8 (end of treatment [EOT]/early termination [ET])

### Overall Study Design

This is an 8-week, outpatient, multicenter, open-label, single-group, flexible-dose study designed to evaluate the safety and tolerability, as well as effectiveness, of switching clinically stable adult subjects with schizophrenia from a typical or atypical antipsychotic to SEP-363856. This study is projected to enroll approximately 120 subjects into a single treatment group (SEP-363856). Over the 8-week treatment period, subjects receive flexibly dosed SEP-363856 (50 to 100 mg/day) and the investigator has the discretion to discontinue each subject's pre-switch antipsychotic treatment by the end of Week 2, 3, 4, 5, or 6. Appropriate duration of pre-switch antipsychotic down-taper for individual subjects is also provided.

Once the pre-switch antipsychotic has been fully discontinued, subjects continue to receive SEP-363856 flexible 50 to 100 mg/day until study endpoint, at the end of Week 8. Study drug is taken at the same time each evening at bedtime and is taken with or without food.

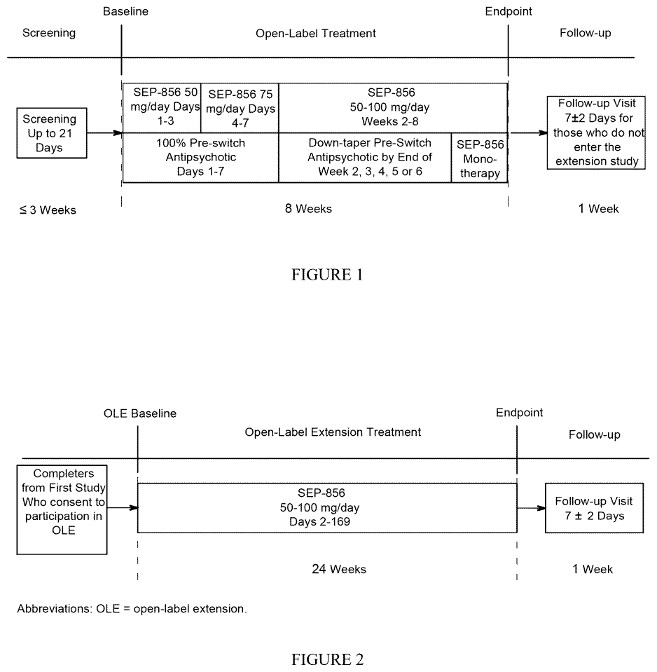

A schematic of the study design is provided in FIG. 1. The study consists of three periods: Screening/Washout (up to 21 days), Treatment (8 weeks) and a Follow-up visit (7±2 days after the last dose of study drug for those subjects who discontinue prior to the Week 8 visit or who complete the study but do not enroll in the open label extension study described in Example 3.

Subjects are evaluated for eligibility during a Screening Period of up to 21 days, during which they continue on their pre-switch antipsychotic medication while being tapered off all other psychotropic medications (except as noted in the Concomitant Medications section below) in a manner that is consistent with labeling recommendations and conventional medical practices. Subjects who are being treated with two antipsychotic medications are permitted to enter the Screening Period. At the start of Screening, the Investigator determines which antipsychotic medication is considered “primary”, and the other one is tapered off during the Screening Period.

### Open-Label Treatment Period (8 Weeks):

At Baseline (Day 1) (pre-switch [PS] Baseline), subjects who have successfully completed Screening and have met the eligibility criteria begin open-label treatment with SEP-363856 while continuing to take the full dose of their pre-switch (PS) antipsychotic. SEP-363856 dosing begins the evening of the PS Baseline visit and continues once-daily, in the evening at bedtime, for the remainder of the Treatment Period, during which study procedures are conducted. Subjects are evaluated at PS Baseline and weekly throughout the rest of the study.

Subjects receive SEP-363856 50 mg/day from Day 1 through Day 3 and remain on 75 mg/day from Day 4 through Day 7. Every effort is made to maintain subjects at 75 mg/day from Day 4 through Day 7. An attempt is made to maintain subjects at 75 mg/day from Day 8 through the end of the 8-week, open-label treatment period. However, beginning on Day 8, the dose of SEP-363856 is adjusted in increments of 25 mg among 3 dose levels (50, 75 and 100 mg), if deemed clinically necessary by the Investigator according to the following predefined requirements:
The SEP-363856 dose is increased to 100 mg/day on Day 8 to help ensure adequate coverage of symptoms, provided there are no significant tolerability problems as judged by the Investigator. Dose increases are made no more frequently than weekly to the next highest dose level (in 25 mg increments). Increases in dose occur at regularly scheduled study visits, when possible.
SEP-363856 dose reductions are made to the next lowest dose level (in 25 mg increments) at any time beginning on Day 8 for safety and intolerability issues as judged by the Investigator.

Beginning on Day 8, the Investigator is permitted to start down-tapering the subject's pre-switch antipsychotic. Discontinuation of the pre-switch antipsychotic is completed by the end of Weeks 2, 3, 4, 5 or 6. Once the pre-switch antipsychotic is fully discontinued, subjects remain on SEP-363856 (50 to 100 mg/day) until the end of Week 8. Subjects who complete the 8-week Open-label Treatment Period are eligible to participate in the open-label extension study described in Example 3. Subjects who early terminate (ET) are not eligible to enroll in the extension study.

### Subject Inclusion Criteria

To qualify for participation, subjects must meet all of the following select inclusion criteria:
Male or female subject between 18 to 65 years of age (inclusive) at the time of consent.
Subject meets DSM-5 criteria for a diagnosis of schizophrenia as established by clinical interview (using the DSM-5 as a reference and confirmed using the Structured Clinical Interview for DSM 5, Clinical Trials Version [SCID-5-CT]). The time since the subject's diagnosis must be ≥1 year prior to Screening.
Subject must have a CGI-S score ≤4 at Screening and Baseline.
Subject must have a PANSS total score ≤80 at Screening and Baseline.
Subject is judged to be clinically stable (i.e., no evidence of an acute exacerbation of schizophrenia) by the Investigator for at least 8 weeks prior to Baseline.
Subject must not have been hospitalized for psychiatric illness for at least 8 weeks prior to Screening.
Subject must be judged by the Investigator to be an appropriate candidate for switching current antipsychotic medication due to safety or tolerability concerns and/or insufficient efficacy.
Subject is taking an oral antipsychotic and the antipsychotic regimen has been stable for at least 6 weeks prior to Screening.
Subjects taking two antipsychotic medications (but not more) at screening are eligible for study inclusion, provided the total daily dose is equivalent to ≤12 mg/day haloperidol. However, the antipsychotic medication determined to be “secondary”, based on investigator judgment, must be discontinued prior to receiving SEP-363856. All subjects must be on a single antipsychotic medication at study baseline (Day 1).
Subject's body mass index (BMI) must be 18 kg/m² to 40 kg/m² (inclusive) at Screening.
Subject is, in the opinion of the Investigator, generally healthy based on screening medical history, physical examination (PE), neurological examination, vital signs, electrocardiogram (ECG) and clinical laboratory values (hematology, chemistry and urinalysis).

### Example 3. An Open-Label Extension Study to Assess the Safety and Tolerability of SEP-363856 in Subjects with Schizophrenia Switched from Typical or Atypical Antipsychotic Agents

This is an open-label 24-week extension study to assess the safety and tolerability of SEP-363856 in subjects with schizophrenia switched from typical or atypical antipsychotic agents, designed to evaluate the effectiveness, safety, and tolerability of switching clinically stable outpatients with schizophrenia, who can potentially benefit from a switch for tolerability or efficacy reasons, from their pre-switch antipsychotic medication to SEP-363856. The open-label extension provides additional, long-term safety, tolerability and effectiveness data from subjects who complete the 8-week switch trial described in Example 2.

The objective of this study is to demonstrate that clinically stable outpatients with schizophrenia who have completed a switch from their current antipsychotic treatment can be safely and effectively maintained on open-label treatment with SEP-363856 for a period of 24 weeks. The outcome measures include standard safety measures (e.g., AE's, SAE's, measures of motor function, assessment of suicidality, laboratory assessments, and ECGs). Additional validated effectiveness assessments for schizophrenia (e.g., PANSS, CGI-S) are included for the purpose of providing longer-term effectiveness data. Additionally, measures assessing function, medication satisfaction, quality of life, sleep, and healthcare utilization are intended to provide information on real-world and overall health aspects associated with transitioning between treatments

### Primary Objective:

To evaluate the long-term safety and tolerability of flexibly dosed SEP-363856 (50, 75, 100 mg/day) in adult subjects with schizophrenia who have completed the study of Example 2 by the incidence of overall adverse events (AEs), serious AEs (SAEs), and AEs leading to discontinuation.

### Other Objectives:

To evaluate the long-term safety and tolerability of SEP-363856 by assessing:
(i) 12-lead electrocardiograms (ECG), (ii) Vital sign measurements, (iii) Clinical laboratory tests, (iv) Columbia—Suicide Severity Rating Scale (C-SSRS), (v) Simpson-Angus Scale (SAS), (vi) Barnes Akathisia Rating Scale (BARS), (vii) Abnormal Involuntary Movement Scale (AIMS)
To evaluate the long-term effectiveness of SEP-363856 using: (i) Positive and Negative Syndrome Scale (PANSS), (ii) Clinical Global Impression-Severity (CGI-S) scale, (iii) Clinical Global Impression-Improvement (CGI-I) scale, (iv) Brief Negative Symptom Scale (BNSS)
To evaluate the long-term effects of SEP-363856 on health-related quality of life as measured by the Short Form Health Survey (SF 12)
To evaluate the long-term effects of SEP-363856 on functional capacity as measured by the Personal and Social Performance Scale (PSP)
To evaluate long-term medication satisfaction as measured by the Medication Satisfaction Questionnaire (MSQ)
To evaluate long-term sleep quality as measured by the Pittsburgh Sleep Quality Index (PSQI)
To evaluate the long-term impact of SEP-363856 on healthcare resource utilization (HCRU)

### Primary Endpoint:

The incidence of overall AEs, SAEs, and AEs leading to discontinuation
Other Safety Endpoint (1): Observed values and changes from Baseline of Example 2 study (pre-switch baseline [PS Baseline]) and Baseline of Example 3 study (open-label extension [OLE] Baseline) in clinical laboratory tests (including hematology, chemistry [including but not limited to lipid parameters and Hemoglobin A1c (HbA1c)], and urinalysis)
Other Safety Endpoint (2): Observed values and changes from PS Baseline and OLE Baseline in vital signs (including temperature, body weight, body mass index [BMI], waist circumference, blood pressure [supine and standing], pulse rate [supine and standing] and respiratory rate) and 12-lead ECG parameters
Other Safety Endpoint (3): Frequency of subjects with suicidal ideation and suicidal behavior based on the C-SSRS
Other Safety Endpoint (4): Change from PS Baseline and OLE Baseline in SAS, BARS and AIMS scores
Other Safety Endpoint (5): Change from PS Baseline and OLE Baseline in PSQI scores
Other Endpoints (1): Changes from PS Baseline and OLE Baseline in: (i) PANSS total score and subscale scores (positive, negative, and general psychopathology), (ii) PANSS Marder Factor (five-factor) scores (positive, disorganized, negative, hostility, and depression/anxiety), (iii) Uncorrelated PANSS (seven-factor) Score Matrix (UPSM) (positive, disorganized, negative apathy/avolition, negative deficit of expression, hostility, anxiety, and depression), (iv) CGI-S score, (v) CGI-I score, (vi) BNSS total score, (vii) SF 12 score, (viii) PSP score, (ix) MSQ score
Other Endpoints (2): HCRU (including numbers of physician office visits, emergency room (ER) visits and hospitalizations, length of hospital stays, employment status and average number of hours caregiver spend helping subjects per week)
Other Endpoints (3): Nicotine use

### Overall Study Design

This is a 24-week, outpatient, multicenter, flexible-dose, open-label extension study designed to evaluate the long-term safety and tolerability of SEP-363856 (50 to 100 mg/day) for the treatment of subjects with schizophrenia who have completed Example 2 study treatment period, during which they were switched from a previous antipsychotic treatment to SEP-363856.

A schematic of the study design is provided in FIG. 2. The study consists of two periods: An open-label extension (OLE) Treatment Period (up to 24 weeks), and a Follow-up Period visit at 7±2 days after last study drug dose for subjects who complete the Treatment Period and those who prematurely discontinue from the study. Subjects who meet the entry criteria and choose to enter the extension study transition immediately at the End of Treatment (EOT) visit from the study of Example 2. Subjects who early terminate (ET) from the Example 2 study are not eligible to enroll in this study. The EOT visit from the Example 2 study serves as the OLE Baseline visit for the present study.

Subjects attend a Baseline visit on Day 1 (same day as the EOT visit of the Example 2 study). Subjects are seen at Baseline and then every 4 weeks thereafter up to Week 24. Telephone calls are made by a member of the clinical research staff to the subjects weekly between visits to collect AEs and concomitant medications, as well as to remind subjects about adherence to study drug administration and upcoming visits.

All subjects begin by receiving open-label SEP-363856 at the same dose they were taking upon completion of the Example 2 study. Thereafter, the dose is adjusted within the range of 50 to 100 mg/day, if deemed clinically necessary by the Investigator. Safety and tolerability are monitored throughout the study by collection of physical examination (PE) results, ECGs, vital signs, AEs, and clinical laboratory parameters. Sleep quality is assessed using the PSQI, and suicidality is assessed using the C-SSRS.

Motor function is assessed using the SAS, BARS and AIMS scales. HCRU is also collected in this study. Effectiveness is evaluated using the PANSS total score and subscale scores, as well as CGI-S score and CGI-I score, BNSS score, and PSQI global scores. Function, quality of life, and treatment satisfaction are assessed using the PSP, SF 12, and MSQ.

### Subject Inclusion Criteria

To qualify for participation, subjects must meet all of the following select inclusion criteria:
Subject must give written informed consent and privacy authorization prior to participation in the study and be able to comply with the protocol, in the opinion of the investigator. Separate consent is obtained from a caregiver or legal guardian if required by local law.
Subject has not taken any psychotropic medication other than the study drug, pre-switch antipsychotic and protocol-allowed medications during the Example 2 study.

### REFERENCES CITED

Ayyagari R, Thomason D, Mu F, Philbin M and Carroll B, Association of antipsychotic treatment switching in patients with schizophrenia, bipolar, and major depressive disorders. Journal of Medical Economics (2020) Vol. 23, No. 2, 204-212.
Barnes T R. A rating scale for drug-induced akathisia. Br J Psychiatry. 1989; 154:672 676.
Barnes T R E. The Barnes Akathisia Rating Scale-Revisited. J Psychopharmacology 2003; 17(4): 365-370.
Buckley P F. Receptor-binding profiles of antipsychotics: clinical strategies when switching between agents. J Clin Psychiatry. 2007; 68 Suppl 6:5-9.
Cerovecki A, Musil R, Klimke A, Seemüller F, Haen E, Schennach R, Kühn K U, Volz H P, Riedel M. Withdrawal symptoms and rebound syndromes associated with switching and discontinuing atypical antipsychotics: theoretical background and practical recommendations. CNS Drugs. 2013 July; 27(7):545-72.
Faries D. Clinical and economic ramifications of switching antipsychotics in the treatment of schizophrenia. BMC Psychiatry. 2009; 9(4):1-9.
First M B, Williams J B W, Karg R S, Spitzer R L. Structured Clinical Interview for DSM-5-Clinical Trials Version (SCID-5-CT); Arlington, V A, American Psychiatric Association, 2015.
Food and Drug Administration (U.S.). Discussion Document for Patient-Focused Drug Development Public Workshop on Guidance 3: SELECT, DEVELOP OR MODIFY FIT-FOR-PURPOSE CLINICAL OUTCOME ASSESSMENTS (including Appendices) (Published at Patient-Focused Drug Development Guidance: Methods to Identify What is Important to Patients and Select, Develop or Modify Fit-for-Purpose Clinical Outcome Assessments, Meeting Oct. 15-16, 2018) (“FDA 2018”)
Food and Drug Administration (U.S.). Patient-Focused Drug Development: Methods to Identify What Is Important to Patients Guidance for Industry, Food and Drug Administration Staff, and Other Stakeholders (October 2019) (“FDA 2019”)
Food and Drug Administration (U.S.). Patient-Focused Drug Development: Collecting Comprehensive and Representative Input; Guidance for Industry, Food and Drug Administration Staff, and Other Stakeholders (June 2020) (“FDA 2020”).
Fulone I, Silva M T, Lopes L C, Switching Between Second-Generation Antipsychotics in Patients with Schizophrenia and Schizoaffective Disorder: 10-Year Cohort Study in Brazil. Front. Pharmacol. (2021) 12:638001.
Guy W. ECDEU Assessment Manual for Psychopharmacology—Revised (DHEW Publication No. ADM 76-338). Rockville, M D, U S Department of Health, Education, and Welfare, 1976:218-22.
Kay S R, Opler L A, Fisbein A. Positive and Negative Syndrome Scale Manual. North Tonawanda, N Y: Multi-Health Systems, 1994.
Kinon B J, Basson B R, Gilmore J A, Malcolm S, Stauffer V L. Strategies for switching from conventional antipsychotic drugs or risperidone to olanzapine. J Clin Psychiatry. 2000 November; 61(11):833-40.
Kirkpatrick B, Strauss G P, Nguyen L, et al. The Brief Negative Symptom Scale: Psychometric Properties. Schizophrenia Bulletin. 2011; 37(2):300-305. doi: 10.1093/schbul/sbq059.
Morosini P-L, Magliano L, Brambilla L, Ugolini R, et al. Development, reliability and acceptability of a new version of the DSM-IV Social and Occupational Functioning Assessment Scale (SOFAS) to assess routine social functioning. Acta Psychiatr Scand. 2000; 101:323-329.
Munetz M R, Benjamin S. How to examine subjects using the Abnormal Involuntary Movement Scale. Hospital and Community Psychiatry 1988; 39:1172-1177.
Opler L A, Kay S S R, Lindenmayer J P, Fiszbein A. Structured Clinical Interview for the Positive and Negative Syndrome Scale (SCI-PANSS). Toronto: Multi-Health Systems, 1992.
Ostuzzi G, Vita G, Bertolini F, Tedeschi F, De Luca B, Gastaldon C, Nosé M, Papola D, Purgato M, Del Giovane C, Correll C U and Barbui C, Continuing, reducing, switching, or stopping antipsychotics in individuals with schizophrenia-spectrum disorders who are clinically stable: a systematic review and network meta-analysis. Lancet Psychiatry 2022; 9:614-24.
PCT Patent Publication No. WO2011/069063
PCT Patent Publication No. WO2019/161238
Perkins D O, Stroup T S, Lieberman J A. Psychotic disorders measures. In Handbook of Psychiatric Measures. Washington, D.C.: American Psychiatric Association, 2000 p. 485-513.
Posner K, Oquendo M A, Gould M, Stanley B, Davies M. Columbia Classification Algorithm of Suicide Assessment (C-CASA): classification of suicidal events in the FDA's pediatric suicidal risk analysis of antidepressants. Am J Psychiatry. 2007 July; 164 (7): 1035-1043.
Simpson G N, Angus J W S. A rating scale for extrapyramidal side effects. Acta Psychiatr Scand. 1970; 212(Suppl 44):S11-S19.
Stahl's Essential Psychopharmacology 7th Edition (November 2020).
Takeuchi H, Thiyanavadivel S, Agid O, Remington G. Rapid vs. slow antipsychotic initiation in schizophrenia: A systematic review and meta-analysis. Schizophr Res. 2018 March; 193:29-36.
U.S. Pat. No. 8,710,245, issued Apr. 29, 2014.
Vernon M K, Revicki D A, Awad A G, Dirani R, et al. Psychometric evaluation of the Medication Satisfaction Questionnaire (MSQ) to assess satisfaction with antipsychotic medication among schizophrenia patients. Schizophr Res. 2010; 118:271-278.
Weiden P J, Simpson G M., et al. Effectiveness of switching to ziprasidone for stable but symptomatic outpatients with schizophrenia. J Clin Psychiatry. 2003; 64:580-588.
Weiden P J. Switching antipsychotics: an updated review with a focus on quetiapine. J Psychopharmacology. 2006; 20:104-118.

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1-2. (canceled)

3. A method of switching a human subject being treated with a dose of a neuropsychiatric medication (treatment NM dose) for a neuropsychiatric disorder during a first treatment period to ulotaront comprising:
a) administering to the subject a dose of the neuropsychiatric medication (first NM tapering dose) and a first dose of ulotaront or a pharmaceutically acceptable salt thereof (first ulotaront tapering dose) during a tapering period; and
b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose; and
wherein, optionally, prior to step (a), the subject has been treated with the treatment dose of the neuropsychiatric medication (treatment NM dose) for the disorder during a first treatment period.

4. A method of treating a neuropsychiatric disorder in a human subject in need thereof comprising:
a) administering a tapering dose of a neuropsychiatric medication (first NM tapering dose) and a first dose of ulotaront or a pharmaceutically acceptable salt thereof (first ulotaront tapering dose) during a tapering period; and
b) after the tapering period, administering a final dose of the ulotaront or pharmaceutically acceptable salt thereof (final ulotaront dose) for a therapeutically effective period of time without administering the neuropsychiatric medication, wherein the first ulotaront tapering dose is optionally less than the final ulotaront dose; and
wherein, optionally, prior to step (a), the subject has been treated with a treatment dose of the neuropsychiatric medication (treatment NM dose) for the disorder during a first treatment period.

5-6. (canceled)

7. The method of claim 3, wherein
i) the neuropsychiatric medication fails to adequately-improve the neuropsychiatric disorder; and
ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

8. The method of claim 4, wherein
i) the neuropsychiatric medication fails adequately to improve the neuropsychiatric disorder; and
ii) the ulotaront or pharmaceutically acceptable salt thereof improves the neuropsychiatric disorder to a greater extent than the neuropsychiatric medication, optionally wherein the subject takes the neuropsychiatric medication for at least two weeks during the tapering period.

9. The method of claim 3, wherein
i) the neuropsychiatric medication affects a target receptor in the subject; and
ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

10. A The method of claim 4, wherein
i) the neuropsychiatric medication affects a target receptor in the subject; and
ii) the ulotaront or pharmaceutically acceptable salt reduces the risk of or prevents the subject from suffering one or more withdrawal adverse events associated with discontinuing a medication that affects the target receptor, optionally wherein the subject takes the neuropsychiatric medication for at least two weeks during the tapering period.

11. The method of claim 3, wherein
i) the neuropsychiatric medication produces one or more adverse events in the subject; and
ii) the ulotaront or pharmaceutically acceptable salt thereof does not produce one or more of the adverse events in the subject, optionally wherein the subject takes the neuropsychiatric medication for at least two weeks during the tapering period.

12. The method of claim 4, wherein
i) the neuropsychiatric medication produces one or more adverse events in the subject; and
ii) the ulotaront or pharmaceutically acceptable salt thereof does not produce one or more of the adverse events in the subject, optionally wherein the subject takes the neuropsychiatric medication for at least two weeks during the tapering period.

13. The method of claim 11, wherein the ulotaront or pharmaceutically acceptable salt thereof resolves the one or more adverse events.

14. The method of claim 3, wherein:
a) the neuropsychiatric disorder is schizophrenia;
b) the neuropsychiatric medication fails to improve a PANSS score in the subject; and
c) the ulotaront or pharmaceutically acceptable salt thereof improves a PANSS score in the subject;
or
d) the neuropsychiatric disorder is schizophrenia;
e) the neuropsychiatric medication fails to improve a PANSS score in the subject to a degree of clinical significance; and
f) the ulotaront or pharmaceutically acceptable salt thereof improves a PANSS score in the subject to a degree of clinical significance;
or
g) the neuropsychiatric disorder is schizophrenia;
h) the subject has a PANSS score ≤80, and optionally ≥30, 34, 38, 42, 46, 50, 54, 58, 62, 66, or 70 immediately prior to commencing ulotaront administration; and
i) the subject's PANSS score is improved by ≥10, 12, 14, 16, or 18 points on an absolute basis;
or
i) the neuropsychiatric disorder is schizophrenia;
k) one or more symptoms of the disorder selected from negative symptoms, positive symptoms, disorganized symptoms, hostility symptoms, and depression/anxiety symptoms persist or worsen in the subject during administration of the neuropsychiatric medication; and
l) the ulotaront or pharmaceutically acceptable salt thereof improves one or more of the persistent or worsened symptoms in the subject;
or
m) the neuropsychiatric disorder is schizophrenia;
n) one or more symptoms of the disorder selected from delusions, suspiciousness/persecution, hallucinatory behavior, grandiosity, conceptual disorganization, poor attention, disturbance of volition, lack of judgement and insight, mannerisms and posturing, stereotyped thinking, unusual thought content, preoccupation, difficulty in abstract thinking, disorientation, anxiety, tension, guilt feelings, depression, somatic concern, hostility, uncooperativeness, poor impulse control, excitement, emotional withdrawal, passive/apathetic social withdrawal, active social avoidance, lack of spontaneity and flow of conversation, poor rapport, blunted affect, and motor retardation persist or worsen in the subject during administration of the neuropsychiatric medication; and
o) the ulotaront or pharmaceutically acceptable salt thereof improves one or more of the persistent or worsened symptoms in the subject.

15-16. (canceled)

17. The method of claim 3, wherein:
a) one or more symptoms of the disorder persist or worsen in the subject during administration of the neuropsychiatric medication; and
b) the ulotaront or pharmaceutically acceptable salt thereof improves one or more of the persistent or worsened symptoms in the subject.

18-19. (canceled)

20. The method of claim 3, wherein:
a) the neuropsychiatric disorder is depression;
b) one or more symptoms of the disorder selected from apparent sadness, reported sadness, inner tension, reduced sleep, reduced appetite, concentration difficulties, lassitude, inability to feel, pessimistic thoughts, and suicidal thoughts persist or worsen in the subject during administration of the neuropsychiatric medication; and
c) the ulotaront or pharmaceutically acceptable salt thereof improves one or more of the persistent or worsened symptoms in the subject.

21. The method of claim 3, wherein:
a) the neuropsychiatric disorder is anxiety;
b) one or more symptoms of the disorder selected from anxious mood, tension, fears, insomnia, intellectual, depressed mood, somatic (muscular), somatic (sensory), cardiovascular symptoms, respiratory symptoms, gastrointestinal symptoms, genitourinary symptoms, autonomic symptoms, and anxious behavior persist or worsen in the subject during administration of the neuropsychiatric medication; and
c) the ulotaront or pharmaceutically acceptable salt thereof improves one or more of the persistent or worsened symptoms in the subject.

22. The method of claim 3, wherein the ulotaront or pharmaceutically acceptable salt thereof reduces nicotine use in the subject; or wherein:
a) nicotine use increases during administration of the neuropsychiatric medication; and
b) the ulotaront or pharmaceutically acceptable salt thereof reduces nicotine use in the subject.

23. (canceled)

24. The method of claim 3, wherein:
a) level of functioning in the subject worsens during administration of the neuropsychiatric medication; and
b) the ulotaront or pharmaceutically acceptable salt thereof improves the level of functioning in the subject;
optionally wherein the ulotaront or pharmaceutically acceptable salt thereof improves the level of functioning in the subject to a degree of clinical significance.

25. (canceled)

26. The method of claim 3, wherein:
a) one or more adverse events occur in the subject during administration of the neuropsychiatric medication; and
b) the ulotaront or pharmaceutically acceptable salt thereof improves one or more of the adverse events in the subject;
optionally wherein the adverse events are selected from (a) abnormal involuntary movements as measured by the AIMS (Abnormal Involuntary Movement Scale); (b) akathisia, preferably as measured by the BARS (Barnes Akathisia Rating Scale); (c) suicidality, preferably as measured by the C-SSRS (Columbia-Suicide Severity Rating Scale); (d) Parkinsonism, preferably as measured by the SAS (Simpson Angus Scale); and (e) sleep quality, preferably as measured by the PSQI (Pittsburgh Sleep Quality Index); or
optionally wherein the adverse events are selected from akathisia, blood prolactin abnormal, blood prolactin increased, blood triglycerides increased, body mass index increased, bradykinesia, bruxism, cogwheel rigidity, dermatillomania, diabetes mellitus, drooling, dyskinesia, dyslipidaemia, dyssomnia, dystonia, electrocardiogram QT prolonged, enuresis, excessive eye blinking, extrapyramidal disorder, galactorrhoea, glucose tolerance impaired, glycosuria, hyperkinesia, hyperprolactinaemia, impaired fasting glucose, increased appetite, metabolic syndrome, muscle rigidity, nuchal rigidity, obesity, obsessive-compulsive disorder, oculogyric crisis, oromandibular dystonia, orthostatic hypertension, overweight, pancreatitis chronic, parkinsonian gait, parkinsonism, psychomotor retardation, restless legs syndrome, restlessness, salivary hypersecretion, sedation, sexual dysfunction, tardive dyskinesia, tic, tongue biting, tongue spasm, torticollis, type 2 diabetes mellitus, and weight increase.

27-28. (canceled)

29. The method of claim 3, wherein during and/or after the tapering period, the subject has a reduced risk of or does not experience clinically significant withdrawal adverse events selected from cholinergic withdrawal adverse events, dopaminergic withdrawal adverse events (nigrostriatal), dopaminergic withdrawal adverse events (mesolimbic or striatal), serotoninergic withdrawal adverse events, histaminergic withdrawal adverse events, and adrenergic withdrawal adverse events.

30. The method of claim 3, wherein the neuropsychiatric medication is a cholinergic medication and, during and/or after the tapering period, the subject has a reduced risk of or does not experience one or more cholinergic withdrawal adverse events; or
wherein the neuropsychiatric medication is a cholinergic medication and, during and/or after the tapering period, the subject has a reduced risk of or does not experience cholinergic withdrawal adverse events selected from agitation, insomnia, anxiety, depression, dizziness, light-headedness, tachycardia, nausea, vomiting, salivation, diarrhea, abdominal cramp, tremor, parkinsonism, restlessness, myalgia, rigidity, paresthesia, fear, hallucinations, confusion, disorientation, hypothermia, and sweating; or
wherein the neuropsychiatric medication is a dopaminergic medication and, during and/or after the tapering period, the subject has a reduced risk of or does not experience dopaminergic withdrawal adverse events (nigrostriatal); or
wherein the neuropsychiatric medication is a dopaminergic medication and, during and/or after the tapering period, the subject has a reduced risk of or does not experience dopaminergic withdrawal adverse events (nigrostriatal) selected from withdrawal dyskinesia, parkinsonism, neuroleptic malignant syndrome, and akathisia; or
wherein the neuropsychiatric medication is a dopaminergic medication and, during and/or after the tapering period, the subject has a reduced risk of or does not experience dopaminergic withdrawal adverse events (mesolimbic or striatal); or
wherein the neuropsychiatric medication is a dopaminergic medication and, during and/or after the tapering period, the subject has a reduced risk of or does not experience dopaminergic withdrawal adverse events (mesolimbic or striatal) selected from auditory hallucinations, persecutory delusions, and other psychotic symptoms; or
wherein the neuropsychiatric medication is a serotoninergic medication and, during and/or after the tapering period, the subject has a reduced risk of or does not experience serotoninergic withdrawal adverse events; or
wherein the neuropsychiatric medication is a serotoninergic medication and, during and/or after the tapering period, the subject has a reduced risk of or does not experience serotoninergic withdrawal adverse events selected from flu-like symptoms, sweating, chills, dizziness, light-headedness, tachycardia, paresthesia, electric chock sensations, anxiety, agitation, low mood, insomnia, nightmares, nausea, vomiting, diarrhea, confusion, and decreased concentration;
wherein the neuropsychiatric medication is a histaminergic medication and, during and/or after the tapering period, the subject has a reduced risk of or does not experience histaminergic withdrawal adverse events; or
wherein the neuropsychiatric medication is a histaminergic medication and, during and/or after the tapering period, the subject has a reduced risk of or does not experience histaminergic withdrawal adverse events selected from irritability, insomnia, agitation, depressed affect, loss of appetite, nausea, tremulousness, incoordination, lethargy, and amnesia; or
wherein the neuropsychiatric medication is an adrenergic medication and, during and/or after the tapering period, the subject has a reduced risk of or does not experience adrenergic withdrawal adverse events; or
wherein the neuropsychiatric medication is an adrenergic medication and, during and/or after the tapering period, the subject has a reduced risk of or does not experience adrenergic withdrawal adverse events selected from headache, anxiety, agitation, hypertension, tachycardia, angina, palpitations, risk of myocardial infarction, pre-syncope, tremulousness, and sweating.

31-41. (canceled)

42. The method of claim 3, wherein the ulotaront or pharmaceutically acceptable salt thereof produces a clinically significant improvement in the disorder; or
wherein the neuropsychiatric medication fails to improve one or more symptom of the disorder, and the ulotaront or pharmaceutically acceptable salt thereof produces a clinically significant improvement in one or more of the symptoms; or
wherein the neuropsychiatric medication produces one or more adverse events and the ulotaront or pharmaceutically acceptable salt thereof produces a clinically significant improvement in one or more of the adverse events.

43-44. (canceled)

45. The method of claim 3, wherein:
a) the neuropsychiatric medication fails to produce a clinically significant improvement in the neuropsychiatric disorder; and
b) the ulotaront or pharmaceutically acceptable salt thereof produces a clinically significant improvement in the neuropsychiatric disorder; or wherein:
c) the neuropsychiatric medication fails to produce a clinically significant improvement in one or more symptom(s) of the disorder; and
d) the ulotaront or pharmaceutically acceptable salt thereof produces a clinically significant improvement in the symptom(s); or wherein
e) the neuropsychiatric medication produces one or more moderate or severe adverse events; and
f) the ulotaront or pharmaceutically acceptable salt thereof improves the one or more moderate or severe adverse events; or wherein
g) the neuropsychiatric medication produces one or more severe adverse events; and
h) the ulotaront or pharmaceutically acceptable salt thereof improves the one or more severe adverse events.

46-48. (canceled)

49. The method of claim 3, wherein the neuropsychiatric medication is selected from the group consisting of cholinergic medications, dopaminergic medications, serotoninergic medications, histaminergic medications, and adrenergic medications; or
wherein the neuropsychiatric medication is an anti-psychotic selected from the group consisting of acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprothepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, and zuclopenthixol; or
wherein the neuropsychiatric medication is an antipsychotic selected from the group consisting of amoxapine, amisulpride, aripiprazole, asenapine, blonanserin, brexpiprazole, cariprazine, carpipramine, clocapramine, clorotepine, clotiapine, clozapine, iloperidone, levosulpiride, lumateperone, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

50-52. (canceled)

53. The method of claim 3, wherein the first NM tapering dose is the same as the treatment NM dose; or wherein the first NM tapering dose is the same as the treatment NM dose, further comprising administering a second tapering dose of the neuropsychiatric medication during the tapering period (second NM tapering dose) which is less than the treatment NM dose and the first NM tapering dose.

54. The method of claim 3, wherein the tapering period is 2-6 weeks, the first NM tapering dose is the same as the treatment NM dose during the first week of the tapering period, and a second tapering dose of the neuropsychiatric medication less than the first NM tapering dose (second NM tapering dose) is administered during the remainder of the tapering period.

55. The method of claim 53, wherein the neuropsychiatric medication has a strong binding affinity for muscarinic-cholinergic (M1) and/or histaminergic (H1) receptors, wherein the first NM tapering dose is administered for 1 week and the second NM tapering dose is administered for 3-5 weeks; or
wherein the neuropsychiatric medication is selected from asenapine, olanzapine, and quetiapine, wherein the first NM tapering dose is administered for 1 week and the second NM tapering dose is administered for 3-5 weeks; or
wherein the neuropsychiatric medication has a strong binding affinity for dopaminergic receptors, wherein the first NM tapering dose is administered for 1 week and the second NM tapering dose is administered for 2-4 weeks; or
wherein the neuropsychiatric medication is selected from lurasidone, paliperidone, risperidone, ziprasidone, haloperidol, and other typical antipsychotics, wherein the first NM tapering dose is administered for 1 week and the second NM tapering dose is administered for 2-4 weeks; or
wherein the neuropsychiatric medication has a long half-life, wherein the first NM tapering dose is administered for 1 week and the second NM tapering dose is administered for 1-3 weeks; or
wherein the neuropsychiatric medication is selected from aripiprazole, brexpiprazole, and cariprazine, wherein the first NM tapering dose is administered for 1 week and the second NM tapering dose is administered for 1-3 weeks; or
wherein the treatment NM dose is at the upper end of the recommended daily dose range, wherein the first NM tapering dose is administered for 1 week and the second NM tapering dose is administered for 3-5 weeks; or
wherein the neuropsychiatric medication is a D2 antagonist and the first treatment period exceeds 6 months, 1 year, 2 years, 3 years, or 5 years, wherein the first NM tapering dose is administered for 1 week and the second NM tapering dose is administered for 3-5 weeks.

56-62. (canceled)

63. The method of claim 3, wherein the first ulotaront tapering dose is less than or equal to the final ulotaront dose; or, wherein the first ulotaront tapering dose is less than or equal to the final ulotaront dose further comprising administering a second dose of ulotaront or pharmaceutically acceptable salt thereof during the tapering period (the second ulotaront tapering dose) which is greater than the first ulotaront tapering dose and optionally the same as the final ulotaront dose.

64. (canceled)

65. The method of claim 63, wherein the tapering period is 2-6 weeks, the first ulotaront tapering dose is administered during days 1-3 of the tapering period, the second ulotaront tapering dose is administered during days 4-7 of the tapering period, and a third ulotaront tapering dose which is optionally the same as the final ulotaront dose is administered during the remainder of the tapering period; or
wherein the first ulotaront tapering dose is 50 mg/day, the second ulotaront tapering dose is 75 mg/day, and the final ulotaront dose is 50-100 mg/day.

66. (canceled)

67. The method of claim 3, wherein the neuropsychiatric disorder is selected from the group consisting of schizophrenia, depression, and anxiety; or
wherein the neuropsychiatric disorder is schizophrenia; or
wherein the neuropsychiatric disorder is MDD; or
wherein the neuropsychiatric disorder is GAD.

68-70. (canceled)

71. The method of claim 3, wherein the first tapering ulotaront dose and the final ulotaront dose are independently selected from 10-150 mg/day or 25-100 mg/day or 50-125 mg/day or 50-100 mg/day or 50-75 mg/day, orally administered; or
wherein the first tapering ulotaront dose and the final ulotaront dose are independently selected from 10 mg/day, 15 mg/day, 20 mg/day, 25 mg/day, 50 mg/day, 75 mg/day, 100 mg/day, 125 mg/day, or 150 mg/day, orally administered; or
wherein the first tapering ulotaront dose and the final ulotaront dose are administered once daily in the fed or fasted state.

72-73. (canceled)

74. The method of claim 3, wherein the ulotaront is administered as the hydrochloride salt.

75. The method of claim 3, wherein the subject takes the neuropsychiatric medication for at least two weeks during the tapering period.

76. The method of claim 3, wherein the first NM tapering dose is the same as the treatment NM dose during the first week of the tapering period, and a second tapering dose of the neuropsychiatric medication less than the first NM tapering dose (second NM tapering dose) is administered during the remainder of the tapering period.
